SEQUENCE LISTING (1 sequence)

SEQ ID NO: 1 - protein, 25 aa, Artificial Sequence
  biotinylated peptide derived from Steroid Receptor Coactivator-1
CPSSHSSLTERHKILHRLLQEGSPS

(19) United States
(12) Patent Application Publication
(10) Pub. No.: US 2014/0039007 A1
(43) Pub. Date: Feb. 6, 2014

(54) COMPOSITIONS AND METHODS FOR MODULATING FARNESOID X RECEPTORS

(71) Applicants: David C. Tully, Emeryville, CA (US); Donatella Chianelli, San Diego, CA (US)

(72) Inventors: David C. Tully, Emeryville, CA (US); Donatella Chianelli, San Diego, CA (US)

(21) Appl. No.: 13/993,231

(22) PCT Filed: Nov. 30, 2011
(86) PCT No.: PCT/US11/62738

§ 371 Date: Aug. 7, 2013

Related U.S. Application Data
(60) Provisional application No. 61/425,145, filed on Dec. 20, 2010.

(51) Int. Cl. C07D 413/14; A61K 31/4545; A61K 45/06; A61K 31/454

(52) U.S. Cl. CPC C07D 413/14; A61K 31/454; A61K 31/4545; A61K 45/06
USPC 514/318; 546/209; 514/326; 546/194

(57) ABSTRACT

The present invention relates to compounds of Formula I, a stereoisomer, enantiomer, a pharmaceutically acceptable salt or an amino acid conjugate thereof; wherein variables are as defined herein; and their pharmaceutical compositions, which are useful as modulators of the activity of Farnesiod X receptors (FXR).

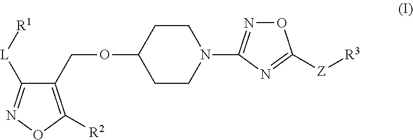

### CROSS-REFERENCE TO RELATED APPLICATIONS

This application claims the benefit of U.S. provisional Ser. No. 61/425,125 filed Dec. 20, 2010, which is incorporated herein in its entirety.

### TECHNICAL FIELD

The present invention relates to compositions and methods for modulating the activity of farnesoid X receptors (FXRs).

### BACKGROUND

The farnesoid X receptor (FXR) is a member of the nuclear hormone receptor superfamily and is primarily expressed in the liver, kidney and intestine (see, e.g., Seol et al. (1995) Mol. Endocrinol. 9:72-85 and Forman et al. (1995) Cell 81:687-693). It functions as a heterodimer with the retinoid X receptor (RXR) and binds to response elements in the promoters of target genes to regulate gene transcription. The FXR-RXR heterodimer binds with highest affinity to an inverted repeat-1 (IR-1) response element, in which consensus receptor-binding hexamers are separated by one nucleotide. FXR is part of an interrelated process, in that FXR is activated by bile acids (the end product of cholesterol metabolism) (see, e.g., Makishima et al. (1999) Science 284: 1362-1365, Parks et al. (1999) Science 284:1365-1368, Wang et al. (1999) MoI. Cell. 3:543-553), which serve to inhibit cholesterol catabolism. See also, Urizar et al. (2000) J. Biol. Chem. 275:39313-39317.

FXR is a key regulator of cholesterol homeostasis, triglyceride synthesis and lipogenesis. (Crawley, Expert Opinion Ther. Patents (2010), 20(8): 1047-1057). In addition to the treatment of dyslipidemia, multiple indications for FXR have been described, including treatment of liver disease, diabetes, vitamin D-related diseases, drug-induced side effects and hepatits. (Crawley, supra). While advances have been made in the development of novel FXR agonists, significant room for improvement remains. It is the object of the present invention to provide novel compounds that are agonists or partial agonists of FXR exhibiting physicochemical, in vitro and/or in vivo ADME (adsorption, distribution, metabolism and excretion) properties superior to known agonists of FXR and/or superior pharmacokinetics in vivo.

### DISCLOSURE OF THE INVENTION

The present invention relates to compositions and methods for modulating the activity of farnesoid X receptors (FXRs). In one aspect, the present invention relates to compounds which act as agonists or partial agonists of FXR.

The compounds of the present invention are defined by Formula I:

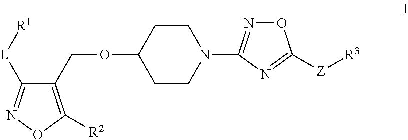

wherein Z is phenyl, C₅₋₇ cycloalkyl or 5-10 membered monocyclic or bicyclic heteroaryl containing 1-2 heteroatoms selected from N, O and S; each of which is optionally substituted with 1-2 R⁶ radicals selected from halogen, C₁₋₆ alkyl, haloC₁₋₆ alkyl, C₁₋₆ alkoxy, haloC₁₋₆ alkoxy, cyclopropyl or NR⁴R⁵;

L is a bond, C₁₋₄ alkylene or C₁₋₄alkylene-O—;

R¹ is phenyl substituted with 1-3 R^{1a}; or R¹ is C₃₋₈ cycloalkyl optionally substituted with 1-3 R^{1a} or phenyl;

R^{1a} is halogen, C₁₋₆ alkyl, haloC₁₋₆ alkyl, C₁₋₆ alkoxy or haloC₁₋₆ alkoxy;

R² is C₁₋₃ alkyl, haloC₁₋₃ alkyl or cyclopropyl optionally substituted with C₁₋₃ alkyl or haloC₁₋₃ alkyl;

R³ is —X—CO₂R⁴, hydroxyC₁₋₆ alkyl, CONR⁴R⁵, CONR(CR₂)₁₋₄CO₂R⁴, CONR(CR₂)₁₋₄SO₃R⁵, cyano, tetrazolyl or SO₂NR⁴R⁵; and

R⁴ and R⁵ are independently hydrogen or C₁₋₆ alkyl; and

X is a bond or C₁₋₄ alkylene; or

a stereoisomer, enantiomer, a pharmaceutically acceptable salt or an amino acid conjugate thereof.

In one embodiment, the invention provides a compound of Formula I, wherein L is a bond, CH₂— or CH₂—O—; and more particularly, wherein L is a bond. In other examples, the invention provides a compound of Formula I, wherein R² is cyclopropyl.

In another embodiment, the invention provides a compound having Formula II:

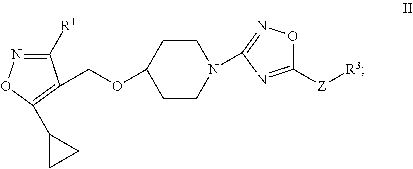

wherein Z is phenyl, thienyl, cyclopentyl, cyclohexyl or pyridyl, each of which is optionally substituted with 1-2 R⁶ radicals selected from halogen, C₁₋₆ alkyl, haloC₁₋₆ alkyl, C₁₋₆ alkoxy, haloC₁₋₆ alkoxy, cyclopropyl or NR⁴R⁵;

R¹ is phenyl substituted with 1-2 R^{1a};

R^{1a} is halogen, C₁₋₆ alkyl, haloC₁₋₆ alkyl, C₁₋₆ alkoxy or haloC₁₋₆ alkoxy;

R³ is —X—CO₂R⁴, hydroxyC₁₋₆ alkyl, CONR⁴R⁵, CONR(CR₂)₁₋₄CO₂R⁴, CONR(CR₂)₁₋₄SO₃R⁵, cyano or tetrazolyl; and

R⁴ and R⁵ are independently hydrogen or C₁₋₆ alkyl; or

a stereoisomer, enantiomer, a pharmaceutically acceptable salt or an amino acid conjugate thereof.

In yet another embodiment, the invention provides a compound having a formula selected from the group consisting of:

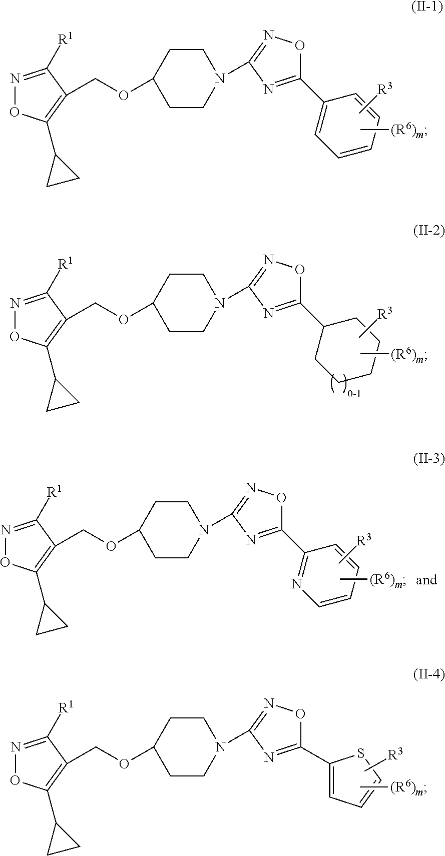

wherein R¹ is phenyl substituted with 1-2 R^{1a};

R^{1a} is halogen, C₁₋₆ alkyl, haloC₁₋₆ alkyl, C₁₋₆ alkoxy or haloC₁₋₆ alkoxy;

R³ is —X—CO₂R⁴, hydroxyC₁₋₆ alkyl, CONR⁴R⁵, CONR(CR₂)₁₋₄CO₂R⁴, CONR(CR₂)₁₋₄SO₃R⁵, cyano or tetrazolyl;

R⁴ and R⁵ are independently hydrogen or C₁₋₆ alkyl;

R⁶ is halogen, C₁₋₆ alkyl, haloC₁₋₆ alkyl, C₁₋₆ alkoxy or haloC₁₋₆ alkoxy; and

m is 0-2; or

a stereoisomer, enantiomer, a pharmaceutically acceptable salt or an amino acid conjugate thereof.

In some embodiments, the invention provides a compound of Formula II-1, II-2, II-3 or II-4, wherein m is 0-2; and R⁶ is selected from fluoro, chloro, bromo, methyl, methoxy, trifluoromethyl or trifluoromethoxy.

In other embodiments, the invention provides a compound of Formula I, II, II-1, II-2, II-3 or II-4, wherein a substituent is defined, collectively or in any combination or sub-combination, as follows:

a) R¹ is phenyl substituted with 1-3 R^{1a}; or R¹ is C₃₋₈ cycloalkyl optionally substituted with 1-3 R^{1a} or phenyl; particularly, R¹ is phenyl, spiro[2.5]octan-6-yl, bicyclo[3.1.0]hexan-6-yl, spiro[2.3]hexan-5-yl, bicyclo[3.1.1]heptan-3-yl, bicyclo[4.1.0]heptan-3-yl, cyclohexyl, cyclopentyl or norbonyl, each of which is optionally substituted with 1-3 R^{1a}; or R¹ is cyclopropyl optionally substituted with 1-2 R^{1a} or phenyl; and more particularly, R¹ is phenyl substituted with 1-2 R^{1a};

b) R^{1a} is halogen, C₁₋₆ alkyl, haloC₁₋₆ alkyl, C₁₋₆ alkoxy or haloC₁₋₆ alkoxy; particularly R^{1a} is from halo, methyl, trifluoromethyl, trifluoromethoxy, difluoromethoxy or methoxy; and more particularly, R¹ is phenyl optionally substituted with 2,6-difluoro, 2-6-dichloro, trifluoromethyl, trifluoromethoxy or difluoromethoxy;

c) R² is C₁₋₃ alkyl, haloC₁₋₃ alkyl or cyclopropyl optionally substituted with C₁₋₃ alkyl or haloC₁₋₃ alkyl; and more particularly, R² is cyclopropyl;

c) R³ is —X—CO₂R⁴, hydroxyC₁₋₆ alkyl, CONR⁴R⁵, CONR(CR₂)₁₄CO₂R⁴, CONR(CR₂)₁₋₄ SO₃R⁵, cyano, tetrazolyl or SO₂NR⁴R⁵; more particularly, R³ is X—CO₂R⁴; X is a bond and each R⁴ and R⁵ are independently hydrogen or C₁₋₆ alkyl;

d) R⁶ is selected halogen, C₁₋₆ alkyl, haloC₁₋₆ alkyl, C₁₋₆ alkoxy, haloC₁₋₆ alkoxy, cyclopropyl or NR⁴R⁵ wherein R⁴ and R⁵ are independently hydrogen or C₁₋₆ alkyl; and more particularly, R⁶ is methyl, methoxy, fluoro or trifluoromethoxy; and

e) m is 0-2; and more particularly, m is 0-1.

In another embodiment, the invention provides a compound selected from the group consisting of:
methyl 4-{3-[4-({5-cyclopropyl-3-[2-(trifluoromethoxy)phenyl]-1,2-oxazol-4-yl}methoxy)piperidin-1-yl]-1,2,4-oxadiazol-5-yl}benzoate;
4-{3-[4-({5-cyclopropyl-3-[2-(trifluoromethoxy)phenyl]-1,2-oxazol-4-yl}methoxy)piperidin-1-yl]-1,2,4-oxadiazol-5-yl}benzoic acid;
methyl 3-{3-[4-({5-cyclopropyl-3-[2-(trifluoromethoxy)phenyl]-1,2-oxazol-4-yl}methoxy)piperidin-1-yl]-1,2,4-oxadiazol-5-yl}benzoate;
3-{3-[4-({5-cyclopropyl-3-[2-(trifluoromethoxy)phenyl]-1,2-oxazol-4-yl}methoxy)piperidin-1-yl]-1,2,4-oxadiazol-5-yl}benzoic acid;
methyl 5-{3-[4-({5-cyclopropyl-3-[2-(trifluoromethoxy)phenyl]-1,2-oxazol-4-yl}methoxy)piperidin-1-yl]-1,2,4-oxadiazol-5-yl}thiophene-2-carboxylate;
5-{3-[4-({5-cyclopropyl-3-[2-(trifluoromethoxy)phenyl]-1,2-oxazol-4-yl}methoxy)piperidin-1-yl]-1,2,4-oxadiazol-5-yl}thiophene-2-carboxylic acid;
methyl 4-[3-(4-({[5-cyclopropyl-3-(2,6-dichlorophenyl)-1,2-oxazol-4-yl]methoxy}piperidin-1-yl)-1,2,4-oxadiazol-5-yl]benzoate;
4-[3-(4-({[5-cyclopropyl-3-(2,6-dichlorophenyl)-1,2-oxazol-4-yl]methoxy}piperidin-1-yl)-1,2,4-oxadiazol-5-yl]benzoic acid;
methyl 3-[3-(4-{[5-cyclopropyl-3-(2,6-dichlorophenyl)-1,2-oxazol-4-yl]methoxy}piperidin-1-yl)-1,2,4-oxadiazol-5-yl]benzoate;
3-[3-(4-{[5-cyclopropyl-3-(2,6-dichlorophenyl)-1,2-oxazol-4-yl]methoxy}piperidin-1-yl)-1,2,4-oxadiazol-5-yl]benzoic acid;
methyl 4-[3-(4-{[5-cyclopropyl-3-(2,6-dichlorophenyl)-1,2-oxazol-4-yl]methoxy}piperidin-1-yl)-1,2,4-oxadiazol-5-yl]cyclohexane-1-carboxylate;
4-[3-(4-{[5-cyclopropyl-3-(2,6-dichlorophenyl)-1,2-oxazol-4-yl]methoxy}piperidin-1-yl)-1,2,4-oxadiazol-5-yl]cyclohexane-1-carboxylic acid;
methyl-3-[3-(4-{[5-cyclopropyl-3-(2,6-dichlorophenyl)-1,2-oxazol-4-yl]methoxy}piperidin-1-yl)-1,2,4-oxadiazol-5-yl]cyclopentane-1-carboxylate;
methyl (1R,3S)-3-[3-(4-{[5-cyclopropyl-3-(2,6-dichlorophenyl)-1,2-oxazol-4-yl]methoxy}piperidin-1-yl)-1,2,4-oxadiazol-5-yl]cyclopentane-1-carboxylate;
methyl (1R,3R)-3-[3-(4-{[5-cyclopropyl-3-(2,6-dichlorophenyl)-1,2-oxazol-4-yl]methoxy}piperidin-1-yl)-1,2,4-oxadiazol-5-yl]cyclopentane-1-carboxylate;
methyl (1S,3S)-3-[3-(4-{[5-cyclopropyl-3-(2,6-dichlorophenyl)-1,2-oxazol-4-yl]methoxy}piperidin-1-yl)-1,2,4-oxadiazol-5-yl]cyclopentane-1-carboxylate;
methyl (1S,3R)-3-[3-(4-{[5-cyclopropyl-3-(2,6-dichlorophenyl)-1,2-oxazol-4-yl]methoxy}piperidin-1-yl)-1,2,4-oxadiazol-5-yl]cyclopentane-1-carboxylate;
3-[3-(4-{[5-cyclopropyl-3-(2,6-dichlorophenyl)-1,2-oxazol-4-yl]methoxy}piperidin-1-yl)-1,2,4-oxadiazol-5-yl]cyclopentane-1-carboxylic acid;
(1R,3S)-3-[3-(4-{[5-cyclopropyl-3-(2,6-dichlorophenyl)-1,2-oxazol-4-yl]methoxy}piperidin-1-yl)-1,2,4-oxadiazol-5-yl]cyclopentane-1-carboxylic acid;
(1R,3R)-3-[3-(4-{[5-cyclopropyl-3-(2,6-dichlorophenyl)-1,2-oxazol-4-yl]methoxy}piperidin-1-yl)-1,2,4-oxadiazol-5-yl]cyclopentane-1-carboxylic acid;
(1S,3S)-3-[3-(4-{[5-cyclopropyl-3-(2,6-dichlorophenyl)-1,2-oxazol-4-yl]methoxy}piperidin-1-yl)-1,2,4-oxadiazol-5-yl]cyclopentane-1-carboxylic acid;
(1S,3R)-3-[3-(4-{[5-cyclopropyl-3-(2,6-dichlorophenyl)-1,2-oxazol-4-yl]methoxy}piperidin-1-yl)-1,2,4-oxadiazol-5-yl]cyclopentane-1-carboxylic acid;
methyl 5-[3-(4-{[5-cyclopropyl-3-(2,6-dichlorophenyl)-1,2-oxazol-4-yl]methoxy}piperidin-1-yl)-1,2,4-oxadiazol-5-yl]thiophene-2-carboxylate;
5-[3-(4-{[5-cyclopropyl-3-(2,6-dichlorophenyl)-1,2-oxazol-4-yl]methoxy}piperidin-1-yl)-1,2,4-oxadiazol-5-yl]thiophene-2-carboxylic acid;
methyl 6-[3-(4-{[5-cyclopropyl-3-(2,6-dichlorophenyl)-1,2-oxazol-4-yl]methoxy}piperidin-1-yl)-1,2,4-oxadiazol-5-yl]pyridine-2-carboxylate;
6-[3-(4-{[5-cyclopropyl-3-(2,6-dichlorophenyl)-1,2-oxazol-4-yl]methoxy}piperidin-1-yl)-1,2,4-oxadiazol-5-yl]pyridine-2-carboxylic acid;
4-[3-(4-{[5-cyclopropyl-3-(2,6-difluorophenyl)-1,2-oxazol-4-yl]methoxy}piperidin-1-yl)-1,2,4-oxadiazol-5-yl]benzoic acid;
4-{3-[4-({5-cyclopropyl-3-[2-(trifluoromethyl)phenyl]-1,2-oxazol-4-yl}methoxy)piperidin-1-yl]-1,2,4-oxadiazol-5-yl}benzoic acid; and
4-{3-[4-({5-cyclopropyl-3-[2-(difluoromethoxy)phenyl]-1,2-oxazol-4-yl}methoxy)piperidin-1-yl]-1,2,4-oxadiazol-5-yl}benzoic acid;

or a stereoisomer, enantiomer, or a pharmaceutically acceptable salt thereof.

In another aspect, the present invention provides pharmaceutical compositions comprising a compound having Formula I, II, II-1, II-2, II-3 or II-4, and a pharmaceutically acceptable carrier. The present invention also provides a pharmaceutical composition comprising a compound of Formula I, II, II-1, II-2, II-3 or II-4 for use in the treatment of a condition mediated by FXR.

The present invention also provides a process for preparing a compound of Formula comprising reacting a compound of Formula III:

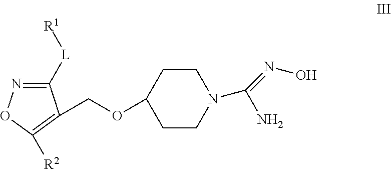

with a compound of Formula R³—Z—C(O)OH to form a compound of Formula IV:

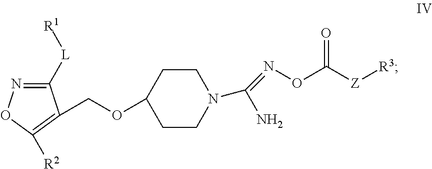

and

converting the compound of Formula IV to a compound of Formula I;

wherein R¹, R², L and Z are as defined for Formula I;

R³ is —X—CO₂R⁴ wherein X is a bond or methylene;

R⁴ is C₁₋₆ alkyl; and optionally, converting a compound of Formula I, wherein the substituents have the meaning as defined in Formula I, into another compound of Formula I as defined herein; and recovering the resulting compound of Formula I in free form or as a salt; and optionally converting the compound of Formula I obtained in free form into a desired salt, or an obtained salt into the free form.

The compounds of Formula I, II, II-1, II-2, II-3 and II-4, and their pharmaceutically acceptable salts exhibit valuable pharmacological properties when tested in vitro in cell-free kinase assays and in cellular assays, and are therefore useful as pharmaceuticals. In particular, the compounds of the invention are agonists of Farnesoid X receptors (FXRs), and are useful as pharmaceuticals to treat FXR-mediated conditions such as cholestasis, intrahepatic cholestatis, estrogen-induced cholestasis, drug-induced cholestasis, cholestasis of pregnancy, parenteral nutrition-associated cholestasis, primary biliary cirrhosis (PBC), primary sclerosing cholangistis (PSC), progressive familiar cholestatis (PFIC), non-alcoholic fatty liver disease (NAFLD), non-alcoholic steatohepatitis (NASH), drug-induced bile duct injury, gallstones, liver cirrhosis, alcohol-induced cirrhosis, cystic fibrosis, bile duct obstruction, cholelithiasis, liver fibrosis, dyslipidemia, atherosclerosis, diabetes, diabetic nephropathy, colitis, newborn jaundice, prevention of kernicterus, venocclusive disease, portal hypertension, metabolic syndrome, hypercholesterolemia, intestinal bacterial overgrowth, erectile dysfunction, progressive fibrosis of the liver caused by any of the diseases above or by infectious hepatitis, or other FXR-mediated conditions leading to extrahepatic cholestasis. The compounds of the invention are also useful for lowering total cholesterol, lowering LDL cholesterol, lowering VLDL cholesterol, raising HDL levels, and/or lowering triglyceride levels.

In one aspect, the invention provides methods for modulating FXR in a cell, comprising contacting the cell with an effective amount of a compound of Formula I, II, II-1, II-2, II-3 or II-4, or a pharmaceutical composition thereof.

In another aspect, the invention provides methods to treat, ameliorate or prevent a FXR-mediated disorder in a subject suffering there from, comprising administering to the subject a therapeutically effective amount of a compound of Formula I, II, II-1, II-2, II-3 or II-4, or a pharmaceutical composition thereof, and optionally in combination with a second therapeutic agent. The present invention also provides for the use of a compound of Formula I, II, II-1, II-2, II-3 or II-4, and optionally in combination with a second therapeutic agent, in the manufacture of a medicament for treating a FXR-mediated disorder such as cholestasis, intrahepatic cholestatis, estrogen-induced cholestasis, drug-induced cholestasis, cholestasis of pregnancy, parenteral nutrition-associated cholestasis, primary biliary cirrhosis (PBC), primary sclerosing cholangistis (PSC), progressive familiar cholestatis (PFIC), non-alcoholic fatty liver disease (NAFLD), non-alcoholic steatohepatitis (NASH), drug-induced bile duct injury, gallstones, liver cirrhosis, alcohol-induced cirrhosis, cystic fibrosis, bile duct obstruction, cholelithiasis, liver fibrosis, dyslipidemia, atherosclerosis, diabetes, diabetic nephropathy, colitis, newborn jaundice, prevention of kernicterus, venocclusive disease, portal hypertension, metabolic syndrome, hypercholesterolemia, intestinal bacterial overgrowth, or erectile dysfunction.

In yet another aspect, the present invention provides a combination comprising a therapeutically effective amount of a compound of Formula I, II, II-1, II-2, II-3 or II-4, and a second therapeutic agent being useful in the treatment of cholestasis, intrahepatic cholestatis, estrogen-induced cholestasis, drug-induced cholestasis, cholestasis of pregnancy, parenteral nutrition-associated cholestasis, primary biliary cirrhosis (PBC), primary sclerosing cholangistis (PSC), progressive familiar cholestatis (PFIC), non-alcoholic fatty liver disease (NAFLD), non-alcoholic steatohepatitis (NASH), drug-induced bile duct injury, gallstones, liver cirrhosis, alcohol-induced cirrhosis, cystic fibrosis, bile duct obstruction, cholelithiasis, liver fibrosis, dyslipidemia, atherosclerosis, diabetes, diabetic nephropathy, colitis, newborn jaundice, prevention of kernicterus, venocclusive disease, portal hypertension, metabolic syndrome, hypercholesterolemia, intestinal bacterial overgrowth, or erectile dysfunction.

### DEFINITIONS

For purposes of interpreting this specification, the following definitions will apply and whenever appropriate, terms used in the singular will also include the plural and vice versa.

As used herein, “C₁₋₆alkyl” denotes a an alkyl radical having from 1 up to 6, particularly up to 4 carbon atoms, the radicals being either linear or branched with single or multiple branching; for example, butyl, such as n-butyl, sec-butyl, isobutyl, tert-butyl; propyl, such as n-propyl or isopropyl; ethyl or methyl; more particularly, methyl, propyl or tert-butyl. “C₁₋₃ alkyl” refers to an alkyl radical as defined herein, containing one to three carbon atoms.

As used herein, the term “alkylene” refers to divalent alkyl group as defined herein above having 1 to 4 carbon atoms. Representative examples of alkylene include, but are not limited to, methylene, ethylene, n-propylene, iso-propylene, n-butylene, sec-butylene, iso-butylene, tert-butylene, and the like.

As used herein, “C₃₋₈ cycloalkyl” refers to saturated or unsaturated monocyclic or bicyclic hydrocarbon groups of 3-8 carbon atoms, and can also include spirocyclic rings. Exemplary monocyclic hydrocarbon groups include, but are not limited to, cyclopropyl, cyclobutyl, cyclopentyl, cyclopentenyl, cyclohexyl and cyclohexenyl and the like. Exemplary bicyclic hydrocarbon groups include but are not limited to bicyclo[2.1.1]hexyl, bicyclo[2.2.1]heptyl, 6,6-dimethylbicyclo[3.1.1]heptyl, 2,6,6-trimethylbicyclo[3.1.1]heptyl, bicyclo[2.2.2]octyl, bicyclo[3.1.0]hexan-6-yl, spiro[2.3]hexan-5-yl, bicyclo[3.1.1]heptan-3-yl, bicyclo[4.1.0]heptan-3-yl, and the like. Exemplary spirocyclic rings include but are not limited to spiro[2.5]octan-6-yl and the like.

As used herein, “C₁₋₆ alkoxy” refers to C₁₋₆ alkyl-O—, and is particularly methoxy, ethoxy, isopropyloxy, or tert-butoxy.

As used herein, “hydroxyC₁₋₆ alkyl” refers to C₁₋₆ alkyl-OH, wherein C₁₋₆ alkyl is as defined above. The hydroxy group may be attached to the alkyl radical on any carbon within the alkyl radical, and is particularly hydroxymethyl, 2-hydroxyethyl or 2-hydroxy-2-propyl.

As used herein, “halogen” or “halo” refers to fluoro, chloro, bromo, and iodo; and more particularly, fluoro or chloro.

As used herein, “halo C₁₋₆ alkyl” refers to an alkyl radical, as defined above, that is substituted by one or more halo radicals, as defined above, and is particularly fluoroC₁₋₆ alkyl, more particularly trifluoromethyl.

As used herein, “halo C₁₋₆alkoxy” refers to an alkoxy radical, as defined above, that is substituted by one or more halo radicals, as defined above, and is particularly fluoroC₁₋₆ alkoxy, more particularly, trifluoromethoxy or difluoromethoxy.

As used herein, a “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

As used herein, the term “amino acid conjugate” refers to conjugates of the compound of Formula I, II, II-1, II-2, II-3 and II-4 with any suitable amino acid. Preferably, such suitable amino acid conjugates of the compound of Formula I, II, II-1, II-2, II-3 and II-4 will have the added advantage of enhanced integrity in bile or intestinal fluids. Suitable amino acids include but are not limited to glycine and taurine. Thus, the present invention encompasses the glycine and taurine conjugates of the compound of Formula I, II, II-1, II-2, II-3 and II-4.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

As used herein, the term“therapeutically effective amount” refers to an amount of the compound of formula (I) which is sufficient to achieve the stated effect. Accordingly, a therapeutical effective amount of a compound of formula (I) used in for the treatment of a condition mediated by FXR will be an amount sufficient for the treatment of the condition mediated by FXR.

As used herein, the term “subject” refers to an animal. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subject is “in need of” a treatment if such subject would benefit biologically, medically or in quality of life from such treatment.

As used herein, the term “dyslipidemia” refers to an abonormality in, or abnormal amounts of lipids and lipoproteins in the blood and the disease states resulting, caused by, exacerbated by, or adjunct to such abnormality (see, Dorland's Illutrated Medical Dictionary, 29th edition, W.B. Saunders Publishing Company, New York, N.Y.). Disease states encompassed within the definition of dyslipidemia as used herein include hyperlipidemia, hypertriglyceremia, low plasma HDL, high plasma LDL, high plasma VLDL, liver cholestasis, and hypercholesterolemia.

As used herein, the phrase “diseases related to dyslipidemia” as used herein refers to diseases including but not limited to atherosclerosis, thrombosis, coronary artery disease, stroke, and hypertension. Diseases related to dyslipidemia also include metabolic diseases such as obesity, diabetes, insulin resistance, and complications thereof.

As used herein, the term “cholestasis” refers to any condition in which the flow of bile from the liver is blocked, and may be intrahepatic (i.e., occurring inside the liver) or extrahepatic (i.e., occurring outside the liver).

As used herein, “liver fibrosis” includes liver fibrosis due to any cause, including but not limited to virally-induced liver fibrosis such as that due to hepatitis B and C; exposure to alcohol (alcoholic liver disease), pharmaceutical compounds, oxidative stress, cancer radiation therapy or industrial chemicals; and diseases such as primary biliary cirrhosis, fatty liver, obesity, non-alcoholic steatohepatitis, cystic fibrosis, hemochromatosis, and auto-immune hepatitis.

“FXR agonist” as used herein refers to an agent that directly binds to and upregulates the activity of FXR.

As used herein, the term “a,” “an,” “the” and similar terms used in the context of the present invention (especially in the context of the claims) are to be construed to cover both the singular and plural unless otherwise indicated herein or clearly contradicted by the context.

The chemical naming protocol and structure diagrams used herein employ and rely on the chemical naming features as utilized by the ChemDraw program (available from CambridgeSoft Corp., Cambridge, Mass.). In particular, compound structures and names were derived using Chemdraw Ultra (Version 10.0) and/or ChemAxon Name Generator (JChem Version 5.3.1.0).

### MODES OF CARRYING OUT THE INVENTION

The present invention relates to compositions and methods for FXR. Various embodiments of the invention are described herein. It will be recognized that features specified in each embodiment may be combined with other specified features to provide further embodiments.

The compounds of the present invention are defined by Formula I:

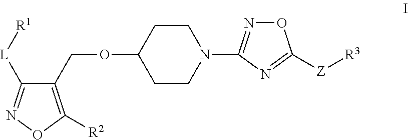

wherein Z is phenyl, C₅₋₇ cycloalkyl or 5-10 membered monocyclic or bicyclic heteroaryl containing 1-2 heteroatoms selected from N, O and S; each of which is optionally substituted with 1-2 R⁶ radicals selected from halogen, C₁₋₆ alkyl, haloC₁₋₆ alkyl, C₁₋₆ alkoxy, haloC₁₋₆ alkoxy, cyclopropyl or NR⁴R⁵;

L is a bond, C₁₋₄ alkylene or C₁₋₄alkylene-O—;

R¹ is phenyl substituted with 1-3 R^{1a}; or R¹ is C₃₋₈ cycloalkyl optionally substituted with 1-3 R^{1a} or phenyl;

R^{1a} is halogen, C₁₋₆ alkyl, haloC₁₋₆ alkyl, C₁₋₆ alkoxy or haloC₁₋₆alkoxy;

R² is C₁₋₃ alkyl, haloC₁₋₃ alkyl or cyclopropyl optionally substituted with C₁₋₃ alkyl or haloC₁₋₃ alkyl;

R³ is —X—CO₂R⁴, hydroxyC₁₋₆ alkyl, CONR⁴R⁵, CONR(CR₂)₁₋₄CO₂R⁴, CONR(CR₂)₁₋₄ SO₃R⁵, cyano, tetrazolyl or SO₂NR⁴R⁵; and

R⁴ and R⁵ are independently hydrogen or C₁₋₆ alkyl; and

X is a bond or C₁₋₄ alkylene; or

a stereoisomer, enantiomer, a pharmaceutically acceptable salt or an amino acid conjugate thereof.

In another embodiment, the invention provides a compound having Formula II:

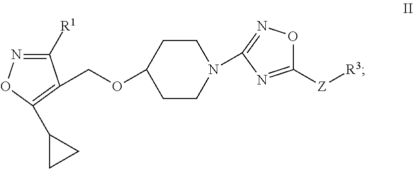

wherein Z is phenyl, thienyl, cyclopentyl, cyclohexyl or pyridyl, each of which is optionally substituted with 1-2 R⁶ radicals selected from halogen, C₁₋₆ alkyl, haloC₁₋₆ alkyl, C₁₋₆ alkoxy, haloC₁₋₆alkoxy, cyclopropyl or NR⁴R⁵;

R¹ is phenyl substituted with 1-2 R^{1a};

R^{1a} is halogen, C₁₋₆ alkyl, haloC₁₋₆ alkyl, C₁₋₆ alkoxy or haloC₁₋₆alkoxy;

R³ is —X—CO₂R⁴, hydroxyC₁₋₆ alkyl, CONR⁴R⁵, CONR(CR₂)₁₋₄CO₂R⁴, CONR(CR₂)₁₋₄ SO₃R⁵, cyano or tetrazolyl; and

R⁴ and R⁵ are independently hydrogen or C₁₋₆ alkyl; or

a stereoisomer, enantiomer, a pharmaceutically acceptable salt or an amino acid conjugate thereof.

In yet another embodiment, the invention provides a compound having a formula selected from the group consisting of:

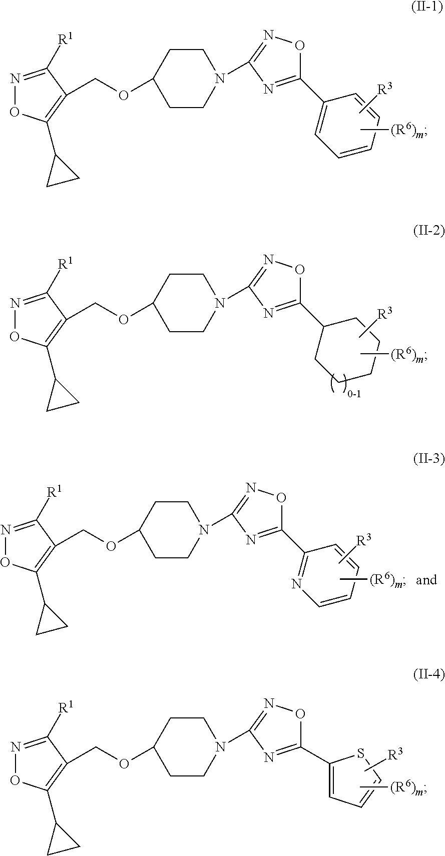

wherein R¹ is phenyl substituted with I-2 R^{1a};

R^{1a} is halogen, C₁₋₆ alkyl, haloC₁₋₆ alkyl, C₁₋₆ alkoxy or haloC₁₋₆alkoxy;

R³ is —X—CO₂R⁴, hydroxyC₁₋₆ alkyl, CONR⁴R⁵, CONR(CR₂)₁₋₄CO₂R⁴, CONR(CR₂)₁₋₄ SO₃R⁵, cyano or tetrazolyl;

R⁴ and R⁵ are independently hydrogen or C₁₋₆ alkyl;

R⁶ is halogen, C₁₋₆ alkyl, haloC₁₋₆ alkyl, C₁₋₆ alkoxy or haloC₁₋₆alkoxy; and

m is 0-2; or

a stereoisomer, enantiomer, a pharmaceutically acceptable salt or an amino acid conjugate thereof.

Unless specified otherwise, the term “compounds of the present invention” refers to compounds of Formula I, II, II-1, II-2, II-3 and II-4, prodrugs thereof, salts of the compound and/or prodrugs, hydrates or solvates of the compounds, salts and/or prodrugs, as well as all stereoisomers (including diastereoisomers and enantiomers), tautomers and isotopically labeled compounds (including deuterium substitutions), as well as inherently formed moieties (e.g., polymorphs, solvates and/or hydrates).

Certain of the compounds described herein contain one or more asymmetric centers or axes and may thus give rise to enantiomers, diastereomers, and other stereoisomeric forms that may be defined, in terms of absolute stereochemistry, as (R)- or (S)-. The present invention is meant to include all possible isomers, including racemic mixtures, optically pure forms and intermediate mixtures. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as 2H, ³H, ¹¹C, ¹³C, ¹⁴C, ¹⁵N, ¹⁸F, ³¹P, ³²P, ³⁵S, ³⁶Cl and ¹²⁵I respectively. The invention includes various isotopically labeled compounds as defined herein, for example those into which radioactive isotopes, such as ³H, ¹³C, and ¹⁴C, are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Isotopically-labeled compounds of Formula I, II, II-1, II-2, II-3 and II-4 can generally be prepared by conventional techniques known to those skilled in the art or by processes analogous to those described in the accompanying Examples and Processes using an appropriate isotopically-labeled reagents in place of the non-labeled reagent previously employed.

Pharmaceutically acceptable solvates in accordance with the invention include those wherein the solvent of crystallization may be isotopically substituted, e.g. D₂O, d⁶-acetone, d⁶-DMSO.

Compounds of the invention, i.e. compounds of Formula I, II, II-1, II-2, II-3 and II-4 that contain groups capable of acting as donors and/or acceptors for hydrogen bonds may be capable of forming co-crystals with suitable co-crystal formers. These co-crystals may be prepared from compounds of Formula I, II, II-1, II-2, II-3 and II-4 by known co-crystal forming procedures. Such procedures include grinding, heating, co-subliming, co-melting, or contacting in solution compounds of Formula I, II, II-1, II-2, II-3 and II-4 with the co-crystal former under crystallization conditions and isolating co-crystals thereby formed. Suitable co-crystal formers include those described in WO 2004/078163. Hence the invention further provides co-crystals comprising a compound of Formula I, II, II-1, II-2, II-3 or II-4.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof. Any resulting mixtures of isomers can be separated on the basis of the physicochemical differences of the constituents, into the pure or substantially pure geometric or optical isomers, diastereomers, racemates, for example, by chromatography and/or fractional crystallization. Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Pharmacology and Utility

The compounds of Formula I, II, II-1, II-2, II-3 and II-4 in free form or in salt form, exhibit valuable pharmacological properties, e.g. FXR modulating properties, e.g. as indicated in in vitro and/or in vivo tests as provided in the next sections, and are therefore indicated for therapy in treating a disorder which may be treated by modulating FXR, such as those described below.

FXR regulates a complex pattern of response genes in the liver that have impact on diverse physiological processes. FXR represses the induction of Cyp7A1 via the upregulation of mRNA encoding SHP, a further nuclear receptor that is dominant repressive over LRH-1. Parallel to the repression of bile acid synthesis via SHP, FXR induces a range of so-called ABC (for ATP-binding cassette) transporters that are responsible for the export of toxic bile acids from the hepatocyte cytosol into the canaliculi, the small bile duct ramifications where the bile originates. This hepatoprotective function of FXR became first apparent with the analysis of FXR knockout mice where under- or overexpression of several ABC-transporters in the liver was shown (Sinai et al., Cell 2000, 102(6), 731-744). Further detailed analysis revealed that the major bile salt excretory pump BSEP or ABCB 11, as well as the key enzyme which mediates lipid transfer from lipoproteins to phospholipids, PLTP, and the two key canalicular membrane transporters for phospholipids, MRP-2 (ABCC4) and MDR-3 (ABCB4), are direct targets for ligand-directed transcriptional activation by FXR. The fact that FXR seems to be the major metabolite sensor and regulator for the synthesis, export and re-circulation of bile acids suggested the use of FXR ligands to induce bile flow and change bile acid composition towards a more hydrophilic composition.

With the development of the first synthetic FXR ligand GW4064 as a tool compound (Maloney et al., J. Med. Chem. 2000, 43(16), 2971-2974; Willson et al., Med. Res. Rev. 2001, 21(6) 513-22), and the development of the semisynthetic artificial bile acid ligand 6-alpha-ethyl-CDCA, the effects of superstimulation of FXR by potent agonists could be analyzed. It was shown that both ligands induce bile flow in bile duct ligated animals. In addition to choleretic effects, hepatoprotective effects could also be demonstrated (Pellicciari et al., J. Med. Chem. 2002, 45(17), 3569-3572; Liu et al., J. Clin. Invest. 2003, 112(11), 1678-1687). This hepatoprotective effect was further narrowed down to an anti-fibrotic effect that results from the repression of Tissue Inhibitors of Matrix-Metalloproteinases, TIMP-1 and 2, the induction of collagen-deposit resolving Matrix-Metalloproteinase 2 (MMP-2) in hepatic stellate cells and the subsequent reduction of alpha-collagen mRNA and Transforming growth factor beta (TGF-beta) mRNA which are both pro-fibrotic factors by FXR agonists (Fiorucci et al., Gastroenterology 2004, 127(5), 1497-1512; Fiorucci et al., Pharmacol. Exp. Ther. 2005, 314(2), 584-595).

The anti-fibrotic activity of FXR is at least partially mediated by the induction of PPARy, a further nuclear receptor, with which anti-fibrotic activity is associated (Fiorucci et al., J. Pharmacol. Exp. Ther. 2005, 315(1), 58-68; GalIi et al., Gastroenterology 2002, 122(7), 1924-1940; Pineda Torra et al., MoI. Endocrinol. 2003, 17(2), 259-272). Furthermore, anti-cholestatic activity was demonstrated in bile-duct ligated animal models as well as in animal models of estrogen-induced cholestasis (Fiorucci et al., J. Pharmacol. Exp. Ther. 2005, 313(2), 604-612).

Genetic studies demonstrate that in hereditary forms of cholestasis (Progressive Familiar Intrahepatic Cholestasis ═PFIC, Type 1-IV), either nuclear localization of FXR itself is reduced as a consequence of a mutation in the FIC1 gene (in PFIC Type I, also called Byler's Disease) (Chen et al., Gastroenterology. 2004, 126(3), 756-64; Alvarez et al., Hum. MoI. Genet. 2004; 13(20), 2451-60) or levels of the FXR target gene encoding MDR-3 phospholipid export pump are reduced (in PFIC Type III). Taken together, there is a growing body of evidence that FXR binding compounds will demonstrate substantial clinical utility in the therapeutic regimen of chronic cholestatic conditions such as Primary Biliary Cirrhosis (PBC) or Primary Sclerosing Cholangitis (PSC) (reviewed in: Rizzo et al., Curr. Drug Targets Immune Endocr. Metabol. Disord. 2005, 5(3), 289-303; Zollner, MoI. Pharm. 2006, 3(3), 231-51, Cai et al., Expert Opin. Ther. Targets 2006, 10(3), 409-421).

Furthermore, FXR seems to be involved in the regulation of many diverse physiological processes which are relevant in the etiology and for the treatment of diseases as diverse as cholesterol gallstones, metabolic disorders such as Type Il Diabetes, dyslipidemias or obesity, chronic inflammatory diseases such as Inflammatory Bowel Diseases or chronic intrahepatic forms of cholestasis and many others diseases (C₁₋audel et al., Arterioscler. Thromb. Vase. Biol. 2005, 25(10), 2020-2030; Westin et al., Mini Rev. Med. Chem. 2005, 5(8), 719-727). Cholesterol gallstones form due to low solubility of cholesterol that is actively pumped out of the liver cell into the lumen of the canaliculi. The relative percentage of the three major components, bile acids, phospholipids and free cholesterol, determines the formation of mixed micelles and hence apparent solubility of free cholesterol in the bile. FXR polymorphisms map as quantitative trait loci as one factor contributing to gallstone disease (Wittenburg, Gastroenterology 2003, 125(3), 868-881). Using the synthetic FXR tool compound GW4064, it could be demonstrated that activation of FXR leads to an improvement of the Cholesterol Saturation Index (CSI) and directly to an abolishment of gallstone formation in C57L gallstone susceptible mice, whereas drug treatment in FXR knockout mice shows no effect on gallstone formation (Moschetta et al., Nature Medicine 2004, 10(12), 1352-1358). These results qualify FXR as a good target for the development of small molecule agonists that can be used to prevent cholesterol gallstone formation or to prevent reformation of gallstones after surgical removal or Shockwave lithotripsy (discussed in: S. Doggrell “New targets in and potential treatments for cholesterol gallstone disease” Curr Opin. Investig. Drugs 2006, 7(4), 344-348).

FXR has also been shown to be a key regulator of serum triglycerides (Maloney et al., J. Med. Chem. 2000, 43(16), 2971-2974; Willson et al., Med. Res. Rev. 2001, 21(6), 513-22). Recent reports indicate that activation of FXR by synthetic agonists leads to significant reduction of serum triglycerides, mainly in the form of reduced VLDL, but also to reduced total serum cholesterol (Kast et al., MoI. Endocrinol. 2001, 15(10), 1720-1728; Urizar et al., Science 2002, 296(5573), 1703-1706; Lambert et al., J. Biol. Chem. 2003, 278, 2563-2570; Watanabe et al., J. Clin. Invest. 2004, 113(10), 1408-1418; Figge et al., J. Biol. Chem. 2004, 279(4), 2790-2799; Bilz et al., Am. J. Physiol. Endocrinol. Metab. 2006, 290(4), E716-22).

However, the lowering of serum triglycerides is not a stand alone effect. Treatment of db/db or ob/ob mice with synthetic FXR agonist GW4064 resulted in marked and combined reduction of serum triglycerides, total cholesterol, free fatty acids, ketone bodies such as 3-OH Butyrate. Moreover, FXR activation engages with the intracellular insulin signaling pathway in hepatocytes, resulting in reduced output of glucose from liver gluconeogenesis but concomitant increase in liver glycogen. Insulin sensitivity as well as glucose tolerance were positively impacted by FXR treatment (Stayrook et al., Endocrinology 2005, 146(3), 984-91; Zhang et al., Proc. Natl. Acad. Sci. USA 2006, 103(4), 1006-1011; Cariou et al., J. Biol. Chem. 2006, 281, 11039-11049; Ma et al., J. Clin. Invest. 2006, 116(4), 1102-1109; Duran-Sandoval et al., Biochimie 2005, 87(1), 93-98).

An effect on reduction of body weight was also recently observed in mice overfed with a high lipid diet (Lihong et al., American Diabetes Association (ADA) 66th annual scientific sessions, June 2006, Abstract Number 856-P). This weight loss effect might result from FXR's induction of FGF-19, a fibroblast growth factor that is known to lead to weight loss and athletic phenotype (Holt et al., Genes Dev. 2003, 17(13), 1581-1591; Tomlinson et al., Endocrinology 2002, 143(5), 1741-1747). Taken together, FXR binding compounds are thought to be good candidates for the treatment of Type Il Diabetes because of their insulin sensitization, glycogenogenic, and lipid lowering effects.

In one embodiment, said compounds and pharmaceutical compositions are used for the preparation of a medicament for the treatment of chronic intrahepatic and some forms of extrahepatic cholestatic conditions, such as primary biliary cirrhosis (PBC), primary sclerosing cholangitis (PSC), progressive familiar cholestasis (PFIC), alcohol-induced cirrhosis and associated cholestasis, or liver fibrosis resulting from chronic cholestatic conditions or acute intraheptic cholestatic conditions such as estrogen or drug induced cholestasis.

In another embodiment, the compounds according to the invention and pharmaceutical compositions comprising said compounds are used in the treatment of Type Il Diabetes which can be overcome by FXR-mediated upregulation of systemic insulin sensitivity and intracellular insulin signalling in liver, increased peripheral glucose uptake and metabolisation, increased glycogen storage in liver, decreased output of glucose into serum from liver-borne gluconeogenesis.

The invention also relates to a compound of Formula I, II, II-1, II-2, II-3 or II-4, or to a pharmaceutical composition comprising said compound, for the treatment of gastrointestinal conditions with a reduced uptake of dietary fat and fat-soluble dietary vitamins which can be overcome by increased intestinal levels of bile acids and phospholipids.

In another embodiment, the compounds according to the invention are useful for beneficially altering lipid profiles, including but not limited to lowering total cholesterol levels, lowering LDL cholesterol levels, lowering VLDL cholesterol levels, raising HDL cholesterol levels, and/or lowering triglyceride levels. Thus, the present invention provides a method for treating FXR mediated conditions such as dyslipidemia and diseases related to dyslipidemia comprising administering a therapeutically effective amount of a compound of the present invention to a subject in need thereof.

In a further embodiment, said compound or pharmaceutical composition is used for treating a disease selected from the group consisting of lipid and lipoprotein disorders such as hypercholesterolemia, hypertriglyceridemia, and atherosclerosis as a clinically manifest condition which can be ameliorated by FXR's beneficial effect on raising HDL cholesterol, lowering serum triglycerides, increasing conversion of liver cholesterol into bile acids and increased clearance and metabolic conversion of VLDL and other lipoproteins in the liver.

In one further embodiment, said compound and pharmaceutical composition are used for the preparation of a medicament where the combined lipid lowering, anti-cholestatic and anti-fibrotic effects of FXR-targeted medicaments can be exploited for the treatment of liver steatosis and associated syndromes such as non-alcoholic steatohepatitis (“NASH”), or for the treatment of cholestatic and fibrotic effects that are associated with alcohol-induced cirrhosis, or with viral-borne forms of hepatitis.

In conjunction with the hypolipidemic effects, it was also shown that loss of functional FXR leads to increased atherosclerosis in ApoE knockout mice (Hanniman et al., J. Lipid Res. 2005, 46(12), 2595-2604). Therefore, FXR agonists might have clinical utility as anti-atherosclerotic and cardioprotective drugs. The downregulation of Endothelin-1 in Vascular Smooth Muscle Cells might also contribute to such beneficial therapeutic effects (He et al., Circ. Res. 2006, 98(2), 192-9).

The invention also relates to a compound according to Formula I, II, II-1, II-2, II-3 or II-4, or a pharmaceutical composition comprising said compound for preventive and posttraumatic treatment of cardiovascular disorders such as acute myocardial infarction, acute stroke, or thrombosis which occur as an endpoint of chronic obstructive atherosclerosis. In a few selected publications, the effects of FXR and FXR agonists on proliferation of cancer and non-malignant cells and apoptosis have been assessed. From these preliminary results it seems as if FXR agonists might also influence apoptosis in cancer cell lines (Niesor et al., Curr. Pharm. Des. 2001, 7(4), 231-59) and in Vascular Smooth Muscle Cells (VSMCs) (Bishop-Bailey et al., Proc. Natl. Acad. Sci. USA. 2004, 101(10), 3668-3673).

Furthermore, FXR seems to be expressed in metastasizing breast cancer cells and in colon cancer (Silva, J. Lipid Res. 2006, 47(4), 724-733; De Gottardi et al., Dig. Dis. Sci. 2004, 49(6), 982-989). Other publications that focus primarily on FXR's effect on metabolism draw a line to intracellular signaling from FXR via the Forkhead/Wingless (FOXO) family of transcriptional modulators to the Phosphatidylinositol-trisphosphat (PI3)-Kinase/Akt signal transduction pathway (Duran-Sandoval et al., J. Biol. Chem. 2005, 280(33), 29971-29979; Zhang et al., Proc. Natl. Acad. Sci. USA. 2006, 103(4), 1006-1011) that is similarly employed by insulin intracellular signaling as well as neoplastically transformed cells. Thus, FXR may also be a potential target for the treatment of proliferative diseases, especially metastasizing cancer forms that overexpress FXR or those where the FOXO/PI3-Kinase/Akt Pathway is responsible for driving proliferation. Therefore, the compounds according to formula (I) or pharmaceutical composition comprising said compounds are suitable for treating non-malignant hyperproliferative disorders such as increased neointima formation after balloon vessel dilatation and stent application due to increased proliferation of vascular smooth muscle cells (VSMCs) or Bening Prostate Hyperplasia (BPH), a pre-neoplastic form of hyperproliferation, other forms of scar tissue formation and fibrotisation which can be overcome by e.g. FXR-mediated intervention into the PI-3Kinase/AKT/mTOR intracellular signalling pathway, reduction in Matrix-Metalloproteinase activity and alpha-Collagen deposition.

In a further embodiment, said compounds and pharmaceutical compositions are used for the treatment of malignant hyperproliferative disorders such as cancer (e.g. certain forms of breast or prostate cancer) where interference with PI-3-Kinase/AKT/mTOR signalling and/or induction of p27kip and/or induction of apoptosis will have a beneficial impact.

Finally, FXR seems also to be involved in the control of antibacterial defense in the intestine (lnagaki et al., Proc. Natl. Acad. Sci. USA. 2006, 103(10), 3920-3905) although an exact mechanism is not provided. From these published data, however, one can conclude that treatment with FXR agonists might have a beneficial impact in the therapy of Inflammatory Bowel Disorders (IBD), in particular those forms where the upper (ileal) part of the intestine is affected (e.g. ileal Crohn's disease) because this seems to be the site of action of FXR's control on bacterial growth. In IBD, the desensitization of the adaptive immune response is somehow impaired in the intestinal immune system. Bacterial overgrowth might then be the causative trigger towards establishment of a chronic inflammatory response. Hence, dampening of bacterial growth by FXR-borne mechanisms might be a key mechanism to prevent acute inflammatory episodes. Thus, the invention also relates to a compound according to formula (I) or a pharmaceutical composition comprising said compound for treating a disease related to Inflammatory Bowel Diseases such as Crohn's disease or Colitis ulcerosa. FXR-mediated restoration of intestinal barrier function and reduction in non-commensal bacterial load is believed to be helpful in reducing the exposure of bacterial antigens to the intestinal immune system and can therefore reduce inflammatory responses.

The invention further relates to a compound or pharmaceutical composition for the treatment of obesity and associated disorders such as metabolic syndrome (combined conditions of dyslipidemias, diabetes and abnormally high body-mass index) which can be overcome by FXR-mediated lowering of serum triglycerides, blood glucose and increased insulin sensitivity and FXR-mediated weight loss.

In one embodiment, said compound or pharmaceutical composition is for treating persistent infections by intracellular bacteria or parasitic protozoae such as Mycobacterium spec. (Treatment of Tuberculosis or Lepra), Listeria monocytogenes (Treatment of Listeriosis), Leishmania spec. (Leishmaniosis), Trypanosoma spec. (Chagas Disease; Trypanosomiasis; Sleeping Sickness).

In a further embodiment, the compounds or pharmaceutical composition of the present invention are useful in the preparation of a medicament for treating clinical complications of Type I and Type II Diabetes. Examples of such complications include Diabetic Nephropathy, Diabetic Retinopathy, Diabetic Neuropathies, Peripheral Arterial Occlusive Disease (PAOD). Other clinical complications of Diabetes are also encompassed by the present invention.

Furthermore, conditions and diseases which result from chronic fatty and fibrotic degeneration of organs due to enforced lipid and specifically triglyceride accumulation and subsequent activation of profibrotic pathways may also be treated by applying the compounds or pharmaceutical composition of the present invention. Such conditions and diseases encompass Non-Alcoholic Steatohepatitis (NASH) and chronic cholestatic conditions in the liver, Glomerulosclerosis and Diabetic Nephropathy in the kidney, Macula Degeneration and Diabetic Retinopathy in the eye and Neurodegenerative diseases such as Alzheimer's Disease in the brain or Diabetic Neuropathies in the peripheral nervous system.

Administration and Pharmaceutical Compositions

In another aspect, the present invention provides a pharmaceutical composition comprising a compound of the present invention and a pharmaceutically acceptable carrier. The pharmaceutical composition can be formulated for particular routes of administration such as oral administration, parenteral administration, and rectal administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers, etc.

Typically, the pharmaceutical compositions are tablets or gelatin capsules comprising the active ingredient together with

a) diluents, e.g., lactose, dextrose, sucrose, mannitol, sorbitol, cellulose and/or glycine;

b) lubricants, e.g., silica, talcum, stearic acid, its magnesium or calcium salt and/or polyethyleneglycol; for tablets also

c) binders, e.g., magnesium aluminum silicate, starch paste, gelatin, tragacanth, methylcellulose, sodium carboxymethylcellulose and/or polyvinylpyrrolidone; if desired

d) disintegrants, e.g., starches, agar, alginic acid or its sodium salt, or effervescent mixtures; and/or

e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein, a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be desirable.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

The present invention further provides anhydrous pharmaceutical compositions and dosage forms comprising the compounds of the present invention as active ingredients, since water may facilitate the degradation of certain compounds. Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The pharmaceutical composition or combination of the present invention can be in unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10-3 molar and 10-9 molar concentrations. A therapeutically effective amount in vivo may range depending on the route of administration, between about 0.1-500 mg/kg, or between about 1-100 mg/kg.

The compound of the present invention may be administered either simultaneously with, or before or after, one or more other therapeutic agent. The compound of the present invention may be administered separately, by the same or different route of administration, or together in the same pharmaceutical composition as the other agents.

In one embodiment, the invention provides a product comprising a compound of Formula I, II, II-1, II-2, II-3 or II-4 and at least one other therapeutic agent as a combined preparation for simultaneous, separate or sequential use in therapy. In one embodiment, the therapy is the treatment of a disease or condition mediated by FXR. Products provided as a combined preparation include a composition comprising a compound of Formula I, II, II-1, II-2, II-3 or II-4, and the other therapeutic agent(s) together in the same pharmaceutical composition, or the compound of Formula I, II, II-1, II-2, II-3 or II-4 and the other therapeutic agent(s) in separate form, e.g. in the form of a kit.

In one embodiment, the invention provides a pharmaceutical composition comprising a compound of Formula I, II, II-1, II-2, II-3 or II-4, and another therapeutic agent(s). It is contemplated that the invention provides a pharmaceutical composition comprising a compound of Formula I, II, II-1, II-2, II-3 or II-4 in combination with a naturally occurring non-toxic bile acid, such as ursodeoxycholic acid, as an aid in preventing possible depletion of fat-soluble vitamins secondary to treatment with an FXR agonist. Accordingly, the compounds of the invention may be administered concurrently with the naturally occurring non-toxic bile acid, either as separate entities or as a single formulation comprising a compound of Formula I, II, II-1, II-2, II-3 or II-4, and naturally occurring bile acid.

Optionally, the pharmaceutical composition may comprise a pharmaceutically acceptable excipient, as described above.

In one embodiment, the invention provides a kit comprising two or more separate pharmaceutical compositions, at least one of which contains a compound of Formula I, II, II-1, II-2, II-3 or II-4. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the invention and the other therapeutic agent may be manufactured and/or formulated by the same or different manufacturers. Moreover, the compound of the invention and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent.

Accordingly, the invention provides the use of a compound of Formula I, II, II-1, II-2, II-3 and II-4 for treating a disease or condition mediated by FXR, wherein the medicament is prepared for administration with another therapeutic agent. The invention also provides the use of another therapeutic agent for treating a disease or condition mediated by FXR, wherein the medicament is administered with a compound of Formula I, II, II-1, II-2, II-3 or II-4.

The invention also provides a compound of Formula I, II, II-1, II-2, II-3 and II-4 for use in a method of treating a disease or condition mediated by FXR, wherein the compound of Formula I, II, II-1, II-2, II-3 or II-4 is prepared for administration with another therapeutic agent.

The invention also provides another therapeutic agent for use in a method of treating a disease or condition mediated by FXR, wherein the other therapeutic agent is prepared for administration with a compound of Formula I, II, II-1, II-2, II-3 or II-4. The invention also provides a compound of Formula I, II, II-1, II-2, II-3 or II-4 for use in a method of treating a disease or condition mediated by FXR, wherein the compound of Formula I, II, II-1, II-2, II-3 or II-4 is administered with another therapeutic agent. The invention also provides another therapeutic agent for use in a method of treating a disease or condition mediated by FXR, wherein the other therapeutic agent is administered with a compound of Formula I, II, II-1, II-2, II-3 or II-4.

The invention also provides the use of a Formula I, II, II-1, II-2, II-3 and II-4 for treating a disease or condition mediated by FXR, wherein the patient has previously (e.g. within 24 hours) been treated with another therapeutic agent. The invention also provides the use of another therapeutic agent for treating a disease or condition mediated by FXR, wherein the patient has previously (e.g. within 24 hours) been treated with a compound of Formula I, II, II-1, II-2, II-3 or II-4.

In one embodiment, the other therapeutic agent is useful in the treatment of dyslipidemia, cholestasis, estrogen-induced cholestasis, drug-induced cholestasis, primary biliary cirrhosis (PBC), primary sclerosing cholangistis (PSC), progressive familiar cholestatis (PFIC), alcohol-induced cirrhosis, cystic fibrosis, cholelithiasis, liver fibrosis, atherosclerosis or diabetes, particularly type II diabetes.

Processes for Making Compounds of the Invention

The present invention also provides a process for the production of a compound of Formula I, comprising reacting a compound of Formula III:

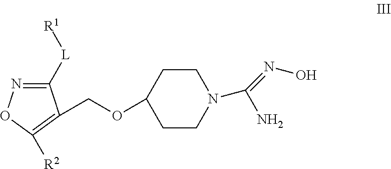

with a compound of Formula R³—Z—C(O)OH to form a compound of Formula IV:

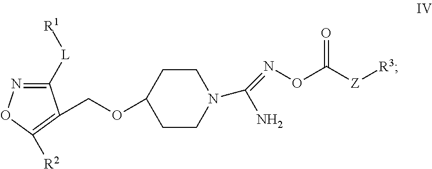

and

converting the compound of Formula IV to a compound of Formula I;

wherein R¹, R² and Z are as defined in Formula I;

R³ is —X—CO₂R⁴ wherein X is a bond or methylene; and

R⁴ is C₁₋₆ alkyl; and

optionally, converting a compound of Formula I, wherein the substituents have the meaning as defined, into another compound of Formula I as defined; and

recovering the resulting compound of Formula I in free form or as a salt; and optionally converting the compound of Formula I obtained in free form into a desired salt, or an obtained salt into the free form.

Each reaction step can be carried out in a manner known to those skilled in the art. For example, a reaction can be carried in the presence of a suitable solvent or diluent or of mixture thereof. A reaction can also be carried, if needed, in the presence of an acid or a base, with cooling or heating, for example in a temperature range from approximately −30° C. to approximately 150° C. In particular examples, a reaction is carried in a temperature range from approximately 0° C. to 100° C., and more particularly, in a temperature range from room temperature to approximately 80° C., in an open or closed reaction vessel and/or in the atmosphere of an inert gas, for example nitrogen.

In one embodiment, the compounds of Formula I can be prepared following the procedures in Scheme 1:

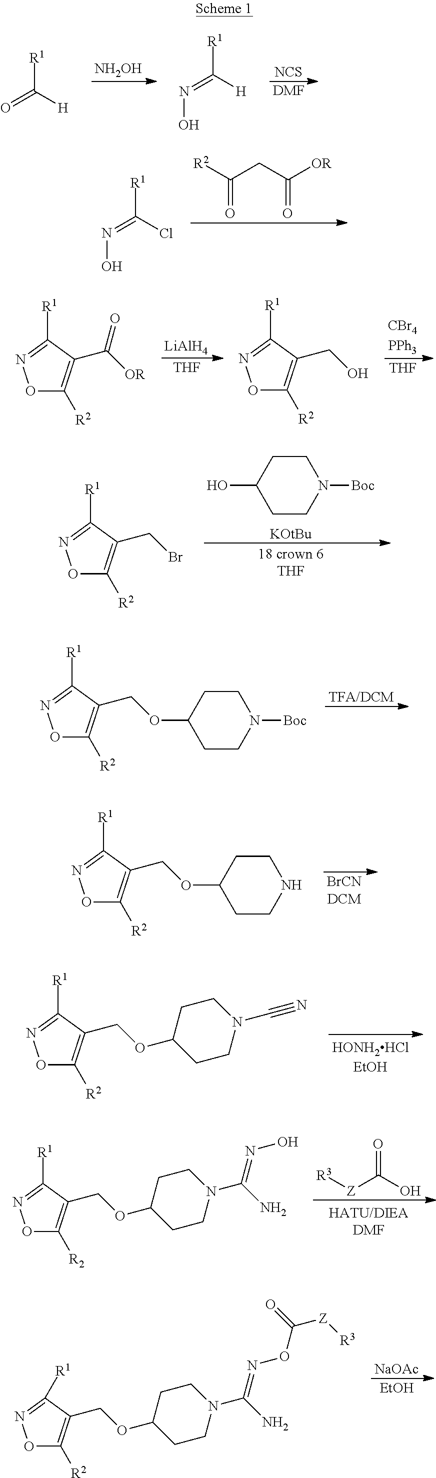

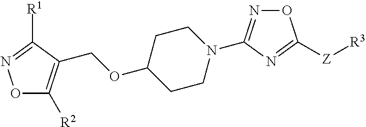

wherein R¹, R² and Z are as defined in Formula I above; R is C₁₋₆ alkyl; and R³ is —X—CO₂R⁴ wherein X is a bond or methylene; and R⁴ is C₁₋₆ alkyl.

The invention also relates to those forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the invention is produced under the process conditions and processed further in situ. Compounds of the invention and intermediates can also be converted into each other according to methods generally known to those skilled in the art. Intermediates and final products can be worked up and/or purified according to standard methods, e.g. using chromatographic methods, distribution methods, (re-) crystallization, and the like.

Within the scope of this text, only a readily removable group that is not a constituent of the particular desired end product of the compounds of the present invention is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosauren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide and Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

All the above-mentioned process steps mentioned herein before and hereinafter can be carried out under reaction conditions that are known to those skilled in the art, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g. in the H⁺ form, depending on the nature of the reaction and/or of the reactants at reduced, normal or elevated temperature, for example in a temperature range of from about −100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers. Mixtures of isomers obtainable according to the invention can be separated in a manner known to those skilled in the art into the individual isomers; diastereoisomers can be separated, for example, by partitioning between polyphasic solvent mixtures, recrystallisation and/or chromatographic separation, for example over silica gel or by e.g. medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofuran or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, methycyclohexane, or mixtures of those solvents, for example aqueous solutions, unless otherwise indicated in the description of the processes. Such solvent mixtures may also be used in working up, for example by chromatography or partitioning.

The compounds of the present invention are either obtained in the free form, as a salt thereof, or as prodrug derivatives thereof. When both a basic group and an acid group are present in the same molecule, the compounds of the present invention may also form internal salts, e.g., zwitterionic molecules. In many cases, the compounds of the present invention are capable of forming acid and/or base salts by virtue of the presence of amino and/or carboxyl groups or groups similar thereto. As used herein, the terms “salt” or “salts” refers to an acid addition or base addition salt of a compound of the invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable.

Salts of compounds of the present invention having at least one salt-forming group may be prepared in a manner known to those skilled in the art. For example, salts of compounds of the present invention having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of the present invention are obtained in customary manner, e.g. by treating the compounds with an acid or a suitable anion exchange reagent. Internal salts of compounds of the present invention containing acid and basic salt-forming groups, e.g. a free carboxy group and a free amino group, may be formed, e.g. by the neutralisation of salts, such as acid addition salts, to the isoelectric point, e.g. with weak bases, or by treatment with ion exchangers. Salts can be converted into the free compounds in accordance with methods known to those skilled in the art. Metal and ammonium salts can be converted, for example, by treatment with suitable acids, and acid addition salts, for example, by treatment with a suitable basic agent.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

The present invention also provides pro-drugs of the compounds of the present invention that converts in vivo to the compounds of the present invention. A pro-drug is an active or inactive compound that is modified chemically through in vivo physiological action, such as hydrolysis, metabolism and the like, into a compound of this invention following administration of the prodrug to a subject. The suitability and techniques involved in making and using pro-drugs are well known by those skilled in the art. Prodrugs can be conceptually divided into two non-exclusive categories, bioprecursor prodrugs and carrier prodrugs. See The Practice of Medicinal Chemistry, Ch. 31-32 (Ed. Wermuth, Academic Press, San Diego, Calif., 2001). Generally, bioprecursor prodrugs are compounds, which are inactive or have low activity compared to the corresponding active drug compound, that contain one or more protective groups and are converted to an active form by metabolism or solvolysis. Both the active drug form and any released metabolic products should have acceptably low toxicity.

Carrier prodrugs are drug compounds that contain a transport moiety, e.g., that improve uptake and/or localized delivery to a site(s) of action. Desirably for such a carrier prodrug, the linkage between the drug moiety and the transport moiety is a covalent bond, the prodrug is inactive or less active than the drug compound, and any released transport moiety is acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of (a) hydroxyl groups with lipophilic carboxylic acids (e.g., a carboxylic acid having at least one lipophilic moiety), or (b) carboxylic acid groups with lipophilic alcohols (e.g., an alcohol having at least one lipophilic moiety, for example aliphatic alcohols).

Exemplary prodrugs are, e.g., esters of free carboxylic acids and S-acyl derivatives of thiols and O-acyl derivatives of alcohols or phenols, wherein acyl has a meaning as defined herein. Suitable prodrugs are often pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the ω-(amino, mono- or di-lower alkylamino, carboxy, lower alkoxycarbonyl)-lower alkyl esters, the α-(lower alkanoyloxy, lower alkoxycarbonyl or di-lower alkylaminocarbonyl)-lower alkyl esters, such as the pivaloyloxymethyl ester and the like conventionally used in the art. In addition, amines have been masked as arylcarbonyloxymethyl substituted derivatives which are cleaved by esterases in vivo releasing the free drug and formaldehyde (Bundgaard, J. Med. Chem. 2503 (1989)). Moreover, drugs containing an acidic NH group, such as imidazole, imide, indole and the like, have been masked with N-acyloxymethyl groups (Bundgaard, Design of Prodrugs, Elsevier (1985)). Hydroxy groups have been masked as esters and ethers. EP 039,051 (Sloan and Little) discloses Mannich-base hydroxamic acid prodrugs, their preparation and use.

Furthermore, the compounds of the present invention, including their salts, may also be obtained in the form of hydrates, or their crystals may, for example, include the solvent used for crystallization. Different crystalline forms may be present. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water. The compounds of the present invention, including salts, hydrates and solvates thereof, may inherently or by design form polymorphs.

Compounds of the invention in unoxidized form may be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

All starting materials, building blocks, reagents, acids, bases, dehydrating agents, solvents and catalysts utilized to synthesize the compounds of the present invention are either commercially available or can be produced by organic synthesis methods known to one of ordinary skill in the art (Houben-Weyl 4th Ed. 1952, Methods of Organic Synthesis, Thieme, Volume 21). All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

### Example 1

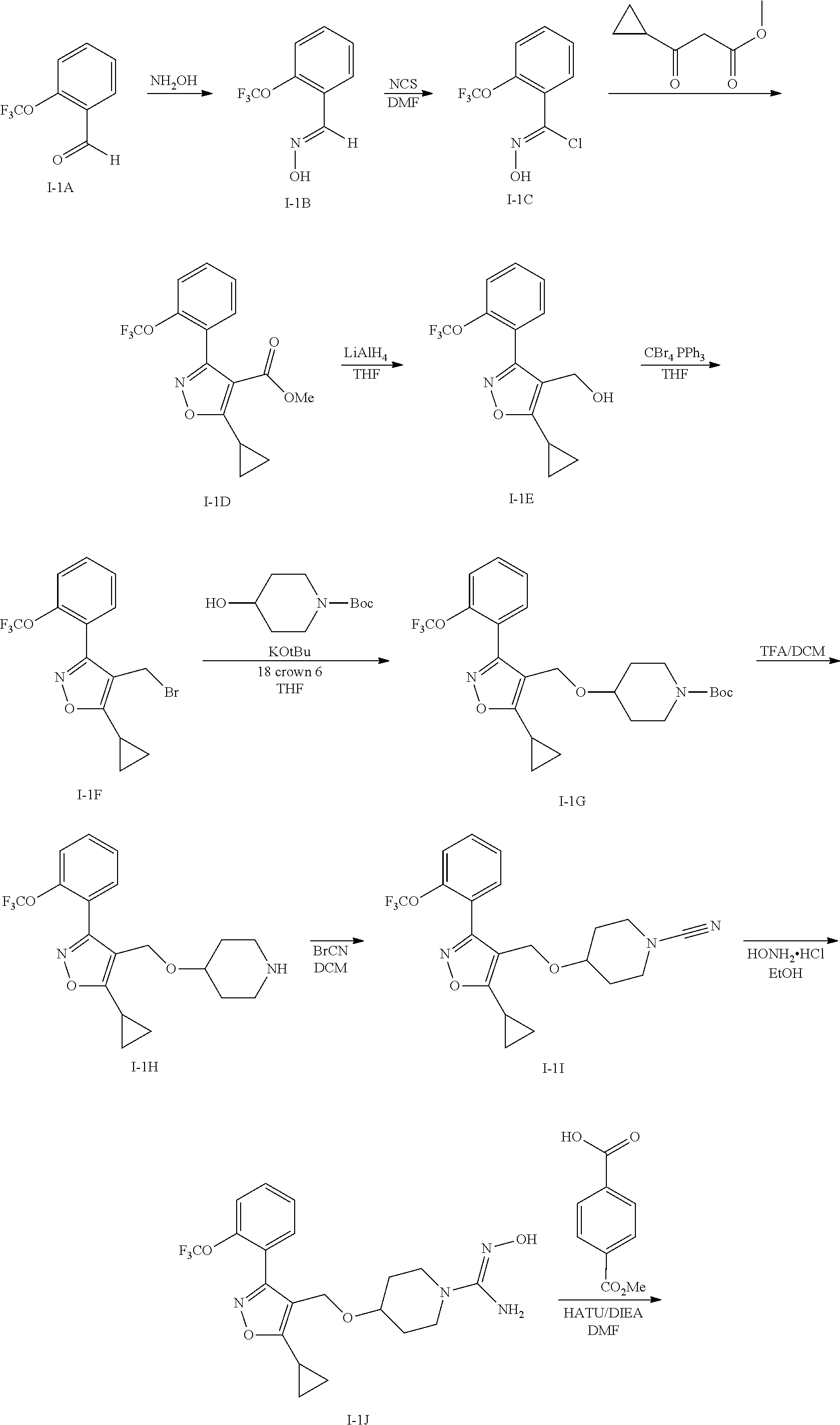

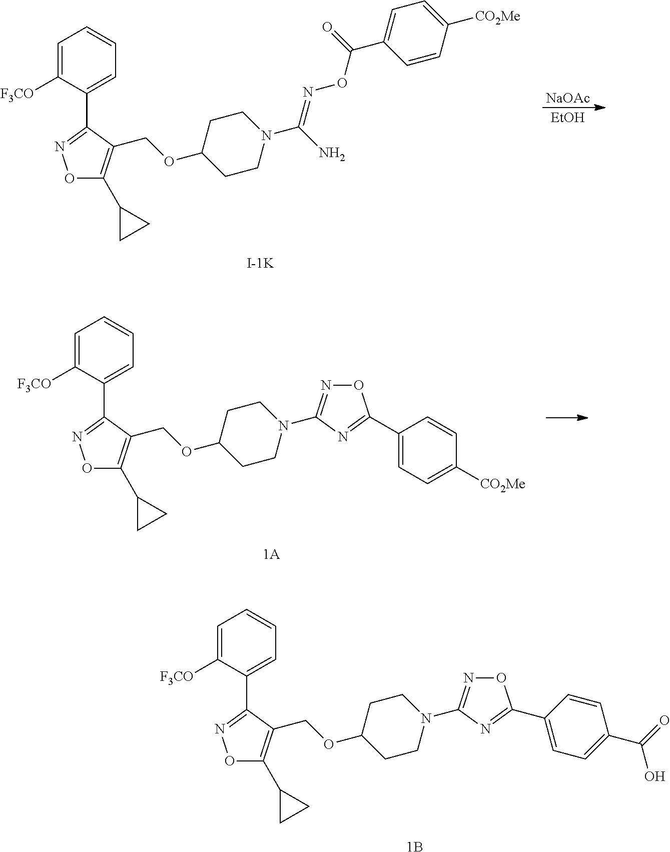

### 2-(trifluoromethoxy)benzaldehyde oxime (I-1B)

A solution of sodium hydroxide (7.00 g, 175.0 mmol, 1.19 equiv) in water (120 mL) was added to a stirred solution of NH₂OH.HCl (11.80 g, 169.8 mmol, 1.15 equiv) in water (120 mL) at 0° C. The resulting solution was stirred for 10 min at 0° C. Then a solution of 2-(trifluoromethoxy)benzaldehyde (28.00 g, 147.3 mmol, 1.00 equiv) in ethanol (120 mL) was added. The resulting solution was allowed to stir for an additional 1 h at room temperature. The resulting solution was diluted with 500 ml of H₂O, extracted with 2×700 mL of ethyl acetate and the organic layers were combined, washed with 2×300 mL of brine, dried over anhydrous sodium sulfate and concentrated under vacuum, which gave (E)-2-(trifluoromethoxy)benzaldehyde oxime as an off-white crystal.

### N-hydroxy-2-(trifluoromethoxy)benzimidoyl chloride (I-1C)

NCS (22.00 g, 166.0 mmol, 1.12 equiv) was added to a stirred solution of (E)-2-(trifluoromethoxy) benzaldehyde oxime (30.00 g, 146.3 mmol, 1.00 equiv) in N,N-dimethylformamide (300 mL) in several batches below 25° C. The resulting solution was stirred for 1 h at room temperature. The resulting solution was diluted with 300 mL of H₂O, extracted with 2×500 mL of ethyl acetate and the organic layers were combined, washed with 5×300 mL of brine, dried over anhydrous sodium sulfate and concentrated under vacuum, which gave (Z)-2-(trifluoromethoxy)benzoyl chloride oxime as a light yellow crystal.

Methyl 5-cyclopropyl-3-(2-(trifluoromethoxy)phenyl)isoxazole-4-carboxylate (I-1D).

Potassium carbonate (11.0 g, 79.7 mmol, 1.09 equiv) was suspended in THF (100 mL). Then the solution of methyl 3-cyclopropyl-3-oxopropanoate (11.0 g, 77.5 mmol, 1.06 equiv) in 50 ml THF was added to the above stirred mixture at −10° C. The resulting solution was stirred for 30 min at −10° C. To this was added a solution of (Z)-2-(trifluoromethoxy)benzoyl chloride oxime (17.6 g, 73.3 mmol, 1.00 equiv) in THF (50 mL) at −5° C. The resulting solution was allowed to stir for 6 hours at 35° C. The resulting solution was diluted with 200 mL of H₂O, extracted with 2×300 mL of ethyl acetate. The organic layer was washed with 2×200 mL of brine, dried over anhydrous sodium sulfate and concentrated under vacuum, then purified by silica gel column with ethyl acetate/petroleum ether (1:100-1:20), which gave methyl 5-cyclopropyl-3-(2-(trifluoromethoxy)phenyl)isoxazole-4-carboxylate as a white solid.

### (5-cyclopropyl-3-(2-(trifluoromethoxy)phenyl)isoxazol-4-yl)-methanol (I-1E)

Into a 250-mL round-bottom flask purged and maintained with an inert atmosphere of nitrogen, was placed a suspension of LiAlH₄ (2.50 g, 65.8 mmol, 2.87 equiv) in tetrahydrofuran (50 mL). This was followed by the addition of a solution of methyl 5-cyclopropyl-3-(2-(trifluoromethoxy)phenyl)isoxazole-4-carboxylate (7.50 g, 22.9 mmol, 1.00 equiv) in tetrahydrofuran (50 mL) dropwise at −10° C. The resulting solution was stirred for 30 min at −10° C. The reaction was then quenched by the addition of 3 mL of ethyl acetate then 3 mL of water and 10 mL of 15% aqueous NaOH. The resulting solution was filtered through celite and the filter cake was washed with 200 mL of ethyl acetate. The filtrate was washed with 2×100 mL of brine, dried over anhydrous sodium sulfate and concentrated under vacuum. This resulted in 7 g of (5-cyclopropyl-3-(2-(trifluoromethoxy)phenyl)isoxazol-4-yl)methanol as yellow oil. (300 MHz, CDCl₃) δ 7.56 (m, 2H), 7.41 (m, 2H), 4.50 (s, 2H), 2.20 (m, 1H), 1.72 (s, 1H, —OH) 1.11-1.28 (m, 4H).

### 4-(bromomethyl)-5-cyclopropyl-3-(2-(trifluoromethoxy)-phenyl)isoxazole (I-1F)

Into a 100 ml round bottom flask was placed (5-cyclopropyl-3-(2-(trifluoromethoxy)phenyl)isoxazol-4-yl)-methanol (4 g, 13.3 mmol) followed by triphenylphosphine (5.6 g, 20 mmol, 1.5 equiv) and dichloromethane (40 mL). The mixture was stirred until completely dissolved, then added to a solution of carbon tetrabromide (6.6 g, 20 mmol, 1.5 eq) in dichloromethane (20 ml) very slowly dropwise. The mixture was stirred for one hour, then the solvent evaporated in vacuo and the residue purified by flash silica chromatography with a 0-50% gradient of ethyl acetate/hexane. The product was obtained as a clear oil. MS m/z 361.9/363.9 (M+1, Br₇₉/Br₈₁ isotope pattern).

### tert-butyl 3-((5-cyclopropyl-3-(2-(trifluoromethoxy)phenyl)isoxazol-4-yl)methoxy)-8-azabicyclo[3.2.1]octane-8-carboxylate (I-1G)

Into a dry 250-mL flask was placed N-Boc-piperidine (1.9 g, 9.5 mmol), 18 crown 6 (2.5 g, 9.5 mmol) and dry THF (50 ml). Potassium tert-butoxide (1.9 g, 2 eq, 19 mmol) was added in portions and the mixture stirred under nitrogen for 1 hour. 4-(bromomethyl)-5-cyclopropyl-3-(2-(trifluoromethoxy)-phenyl)isoxazole (3.01 g, 8.5 mmol) was dissolved in anhydrous THF (50 mL) and added dropwise and the mixture stirred overnight under nitrogen. The solvent was then evaporated in vacuo and the residue suspended in water (50 ml) and ethyl acetate (50 ml). The organic layer was collected and the aqueous layer was extracted with a further wash of ethyl acetate (25 mL). The organics were combined and dried (MgSO₄) then evaporated in vacuo. The oil was purified by flash column chromatography with a 0-100% gradient of ethyl acetate/hexane. The product was obtained as a solid.

### 4-((8-azabicyclo[3.2.1]octan-3-yloxy)methyl)-5-cyclopropyl-3-(2-(trifluoromethoxy)phenyl)isoxazole (I-1H)

Into a 100 ml flask was placed tert-butyl 3-((5-cyclopropyl-3-(2-(trifluoromethoxy)phenyl)isoxazol-4-yl)methoxy)-8-azabicyclo[3.2.1]octane-8-carboxylate (3.29 g, 6.83 mmol) and a solution of dichloromethane (20 ml) and trifluoroacetic acid (10 ml). The mixture was stirred for an hour, the solvent was evaporated in vacuo and the residue suspended in sodium bicarbonate (50 ml saturate aqueous solution) and ethyl acetate (50 ml). The organic layer was collected and the aqueous layer was extracted with a further wash of ethyl acetate (25 ml). The organics were combined and dried (MgSO₄) then evaporated in vacuo. The oil was purified by flash column chromatography with a 0-20% gradient of ethanol/dichloromethane. The product was obtained as a pale yellow oil.

### 4-((5-cyclopropyl-3-(2-(trifluoromethoxy)phenypisoxazol-4-yl)methoxy)piperidine-1-carbonitrile (I-1I)

4-((8-azabicyclo[3.2.1]octan-3-yloxy)methyl)-5-cyclopropyl-3-(2-(trifluoromethoxy)phenyl)isoxazole (200 mg, 0.52 mmol) and potassium carbonate (217 mg, 3 equivalents, 1.56 mmol) was suspended in water (5 ml). Cyanogen bromide (60 mg, 1.1 equivalents, 0.57 mmol) was added dropwise in dichloromethane (5 ml) and the mixture was stirred for two hours. The mixture was then diluted with 0.1M hydrochloric acid (10 ml) and extracted with dichloromethane (2×10 ml). The organics were combined, dried (MgSO₄) and evaporated in vacuo then used directly in the next step.

Intermediate I-J: (E)-4-((5-cyclopropyl-3-(2-(trifluoromethoxy)phenypisoxazol-4-yl)methoxy)-N′-hydroxypiperidine-1-carboximidamide (I-1J).

4-((5-cyclopropyl-3-(2-(trifluoromethoxy)phenyl)isoxazol-4-yl)methoxy)piperidine-1-carbonitrile (0.52 mmol) was dissolved in ethanol (5 ml) and was treated with sodium acetate (43 mg, 1 equivalent, 0.52 mmol) and hydroxylamine hydrochloride (36 mg, 1.1 equivalents, 0.57 mmol) and the mixture stirred for two hours at 60° C. The solvent was evaporated in vacuo and the residue dissolved in ethyl acetate (10 ml) and washed with water (10 ml). The organics were dried (MgSO₄) and evaporated in vacuo. The product was obtained as a clear oil.

### Methyl 4-((((amino(4-((5-cyclopropyl-3-(2-(trifluoromethoxy)phenypisoxazol-4-yl)methoxy)piperidin-1-yl)methylene)amino)oxy)carbonyl)benzoate (I-1K)

(E)-4-((5-cyclopropyl-3-(2-(trifluoromethoxy)phenyl)isoxazol-4-yl)methoxy)-N′-hydroxypiperidine-1-carboximidamide (0.2 mmol) was dissolved in anhydrous N,N-dimethylformamide (2 ml) and was treated with 4-(methoxycarbonyl)benzoic acid (37 mg, 1 equivalent, 0.2 mmol) 2-(7-aza-1H-benzotriazole-1-yl)-1,1,3,3-tetramethyluronium hexafluorophosphate (78 mg, 1 equivalents, 0.2 mmol) and N,N-diisopropylethylamine (0.1 mL, 3 equivalents, 0.61 mmol) the mixture was stirred at room temperature for three hours. The reaction mixture was quenched with water and extracted with ethyl acetate (2×30 mL). The organic layer was washed with water (2×20 mL), brine (1×20 mL) dried over anhydrous sodium sulfate, and was evaporated in vacuo. The title compound was obtained as light brown oil, and was used in the next step without further purification. MS (m/z) (M+1)⁺: 603.2.

### Methyl 4-(3-(4-((5-cyclopropyl-3-(2-(trifluoromethoxy)phenyl)isoxazol-4-yl)methoxy)piperidin-1-yl)-1,2,4-oxadiazol-5-yl)benzoate (1A)

A solution of methyl 4-((((amino(4-((5-cyclopropyl-3-(2-(trifluoromethoxy)phenyl)isoxazol-4-yl)methoxy)piperidin-1-yl)methylene)amino)oxy)carbonyl)benzoate (120 mg, 0.2 mmol) in ethyl alcohol (2 mL) was treated with sodium acetate (170 mg, 10 equivalents, 2 mmol) and was then refluxed for 2 hours. The reaction mixture was cooled to room temperature and diluted with ethyl acetate. The organic was washed with water (2×20 mL) brine (1×20 mL) and dried over anhydrous sodium sulfate. The crude oil was purified by flash column chromatography with a 0-30-% gradient of ethyl acetate/hexane. The product was obtained as a yellow semisolid.

### 4-(3-(4-((5-cyclopropyl-3-(2-(trifluoromethoxy)phenyl)isoxazol-4-yl)methoxy)piperidin-1-yl)-1,2,4-oxadiazol-5-yl)benzoic acid (1B)

Methyl 4-((((amino(4-((5-cyclopropyl-3-(2-(trifluoromethoxy)phenyl)isoxazol-4-yl)-methoxy)piperidin-1-yl)methylene)amino)oxy)carbonyl)benzoate (23 mg, 0.04 mmol) was suspended in a 3:2:1 THF: MeOH:H₂O solution (1 mL) and treated with 6N lithium hydroxide (40 mL, 6 equivalent). The reaction was stirred at room temperature for 14 hours. The reaction mixture was diluted with water (1 mL) and the pH neutralized with 3N hydrochloric acid. A solid was collected by filtration and dried in vacuo to afford the title compound as white solid.

| | | Physical Data |
|---|---|---|
| | | MS (m/z), Elemental |
| Ex. | Structure | Analysis, ¹H NMR |
| 1A | 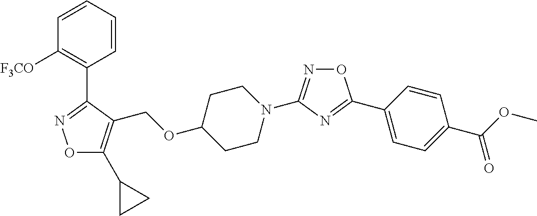 [SMILES: COC(=O)c1ccc(-c2nc(N3CCC(OCc4c(-c5ccccc5OC(F)(F)F)noc4C4CC4)CC3)no2)cc1] | MS m/z 585.2 (M + 1); ¹H NMR (CDCl3, 400 MHz) δ 8.16-8.13 (m, 4H), 7.58-7.49 (m, 2H), 7.40-7.39 (m, 2H), 4.40 (s, 2H), 3.95 (s, 3H), 3.70-3.67 (m, 2H), 3.49-3.47 (m, 1H), 2.24- 2.18 (m, 2H), 2.14-2.12 (m, 1H), 1.80-1.79 (m, 2H), 1.25-1.23 (m, 4H), 1.12-1.10 (m, 2H). |
| 1B | 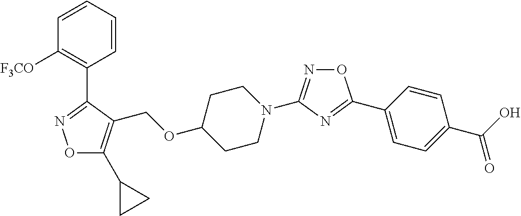 [SMILES: O=C(O)c1ccc(-c2nc(N3CCC(OCc4c(-c5ccccc5OC(F)(F)F)noc4C4CC4)CC3)no2)cc1] | MS m/z 571.2 (M + 1); ¹H NMR (DMSO D₆, 400 MHz) δ 8.12 (s, 4H), 7.68-7.63 (m, 2H), 7.57- 7.52 (m, 2H), 4.37 (s, 2H), 3.52- 3.48 (m, 3H), 3.18-3.13 (m, 2H), 2.35-2.31 (m, 1H), 1.78-1.74 (m, 2H), 1.42-1.39 (m, 2H), 1.14- 1.06 (m, 4H). |

### Examples 2-12

The following compounds were prepared following the procedure in Example 1.

| | | Physical Data |
|---|---|---|
| | | MS (m/z), Elemental |
| Ex. | Structure | Analysis, ¹H NMR |
| 2A | 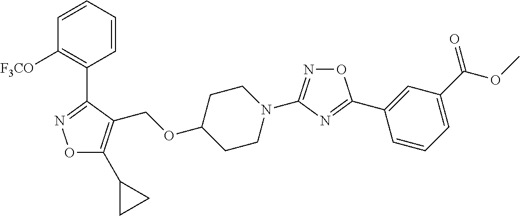 [SMILES: COC(=O)c1cccc(-c2nc(N3CCC(OCc4c(-c5ccccc5OC(F)(F)F)noc4C4CC4)CC3)no2)c1] | MS m/z 585.2 (M + 1); |
| 2B | 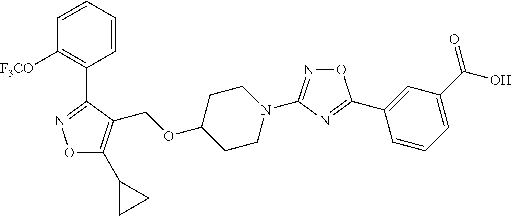 [SMILES: O=C(O)c1cccc(-c2nc(N3CCC(OCc4c(-c5ccccc5OC(F)(F)F)noc4C4CC4)CC3)no2)c1] | MS m/z 571.2 (M + 1); ¹H NMR (DMSOd₆, 400 MHz) δ 8.51 (s, 1H), 8.23-8.17 (m, 2H), 7.74-7.63 (m, 3H), 7.57- 7.32 (m, 2H), 4.38 (s, 2H), 3.55-3.44 (m, 3H), 3.19-3.12 (m, 2H), 2.37-2.31 (m, 1H), 1.79-1.74 (m, 2H), 1.44-1.36 (m, 2H), 1.17-1.06 (m, 4H). |
| 3A | 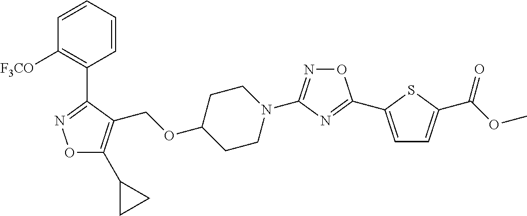 [SMILES: COC(=O)c1ccc(-c2nc(N3CCC(OCc4c(-c5ccccc5OC(F)(F)F)noc4C4CC4)CC3)no2)s1] | MS m/z 591.1 (M + 1); |
| 3B | 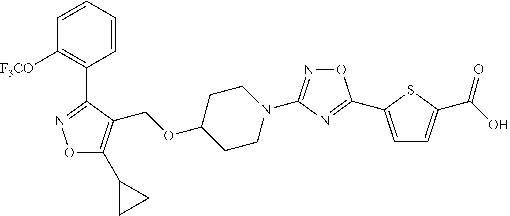 [SMILES: O=C(O)c1ccc(-c2nc(N3CCC(OCc4c(-c5ccccc5OC(F)(F)F)noc4C4CC4)CC3)no2)s1] | MS m/z 577.1 (M + 1); |
| 4A | 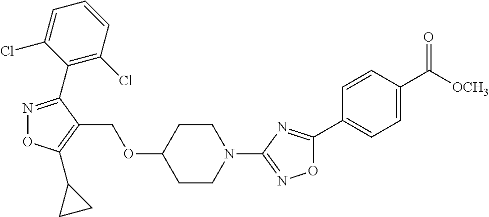 [SMILES: COC(=O)c1ccc(-c2nc(N3CCC(OCc4c(-c5c(Cl)cccc5Cl)noc4C4CC4)CC3)no2)cc1] | MS m/z 569.2 (M + 1); |
| 4B | 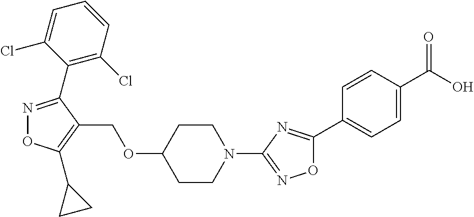 [SMILES: O=C(O)c1ccc(-c2nc(N3CCC(OCc4c(-c5c(Cl)cccc5Cl)noc4C4CC4)CC3)no2)cc1] | MS m/z 551.1 (M + 1); NMR (DMSO D₆, 400 MHz) 8.05 (s, 4H), 7.05-7.56 (m, 2H), 7.49- 7.45 (m, 1H), 4.26 (s, 2H), 3.42-3.23 (m, 3H-region obscured by apparent water resonance), 3.13-3.05 (m, 2H), 2.31-2.26 (m, 1H), 1.68-1.60 (m, 2H), 1.34-1.24 (m, 2H), 1.11-1.01 (m, 4H). |
| 5A | 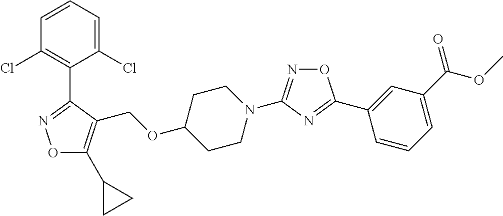 [SMILES: COC(=O)c1cccc(-c2nc(N3CCC(OCc4c(-c5c(Cl)cccc5Cl)noc4C4CC4)CC3)no2)c1] | MS m/z 569.1 (M + 1); |
| 5B | 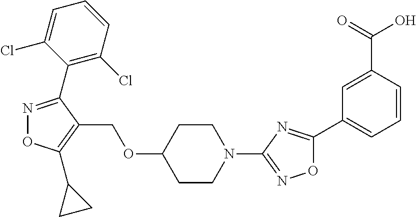 [SMILES: O=C(O)c1cccc(-c2nc(N3CCC(OCc4c(-c5c(Cl)cccc5Cl)noc4C4CC4)CC3)no2)c1] | MS m/z 555.0 (M + 1); ); NMR (DMSO D₆, 400 MHz) 8.51 (s, 1H), 8.25-8.19 (m, 2H), 7.74 (t, J = 8 Hz, 1H), 7.64- 7.62 (m, 2H), 7.56-7.52 (m, 1H), 4.33 (s, 2H), 3.48-3.34 (m, 3H-region obscured by apparent water resonance), 3.21-3.12 (m, 2H), 2.39-2.32 (m, 1H), 1.76-1.68 (m, 2H), 1.41-1.30 (m, 2H), 1.18-1.07 (m, 4H). |
| 6A | 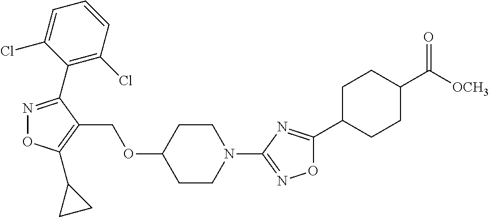 [SMILES: COC(=O)C1CCC(c2nc(N3CCC(OCc4c(-c5c(Cl)cccc5Cl)noc4C4CC4)CC3)no2)CC1] | MS m/z 575.2 (M + 1); |
| 6B | 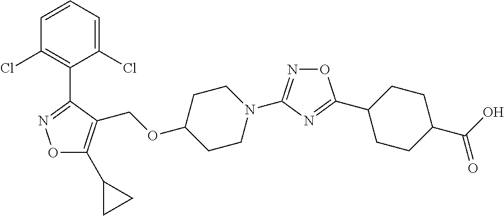 [SMILES: O=C(O)C1CCC(c2nc(N3CCC(OCc4c(-c5c(Cl)cccc5Cl)noc4C4CC4)CC3)no2)CC1] | MS m/z 561.2 (M + 1); |
| 7A | 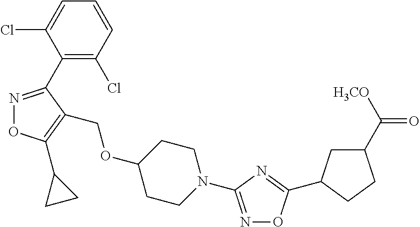 [SMILES: COC(=O)C1CCC(c2nc(N3CCC(OCc4c(-c5c(Cl)cccc5Cl)noc4C4CC4)CC3)no2)C1] | MS m/z 561.2 (M + 1); |
| 7B | 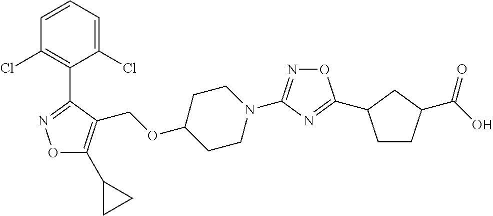 [SMILES: O=C(O)C1CCC(c2nc(N3CCC(OCc4c(-c5c(Cl)cccc5Cl)noc4C4CC4)CC3)no2)C1] | MS m/z 547.2 (M + 1); |
| 8A | 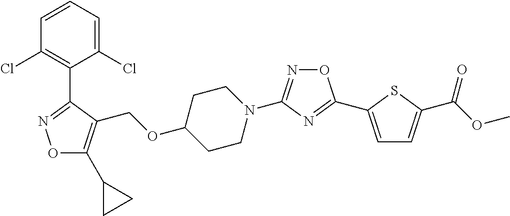 [SMILES: COC(=O)c1ccc(-c2nc(N3CCC(OCc4c(-c5c(Cl)cccc5Cl)noc4C4CC4)CC3)no2)s1] | MS m/z 575.1 (M + 1); |
| 8B | 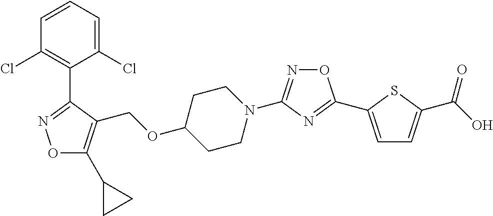 [SMILES: O=C(O)c1ccc(-c2nc(N3CCC(OCc4c(-c5c(Cl)cccc5Cl)noc4C4CC4)CC3)no2)s1] | MS m/z 561.1 (M + 1); ¹H NMR (CDCl3, 400 MHz) δ 7.88 (d, J = 2.4 Hz, 1H), 7.75 (d, J = 4 Hz, 1H), 7.65-7.61 (m, 2H), 7.56-7.52 (m, 1H), 4.32 (s, 2H), 3.47-3.34 (m, 3H- region obscured by apparent water resonance), 3.17-3.06 (m, 2H), 2.38-2.32 (m, 1H), 1.72-1.64 (m, 2H), 1.41-1.30 (m, 2H), 1.18-1.05 (m, 4H). |
| 9A | 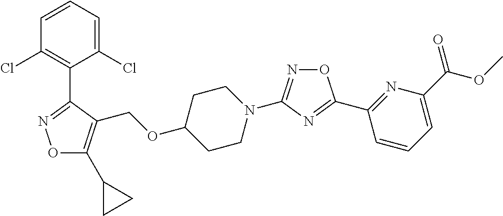 [SMILES: COC(=O)c1cccc(-c2nc(N3CCC(OCc4c(-c5c(Cl)cccc5Cl)noc4C4CC4)CC3)no2)n1] | MS m/z 570.1 (M + 1); |
| 9B | 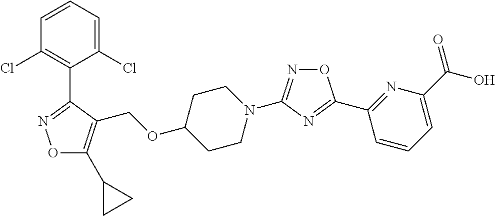 [SMILES: O=C(O)c1cccc(-c2nc(N3CCC(OCc4c(-c5c(Cl)cccc5Cl)noc4C4CC4)CC3)no2)n1] | MS m/z 556.1 (M + 1); |
| 10 | 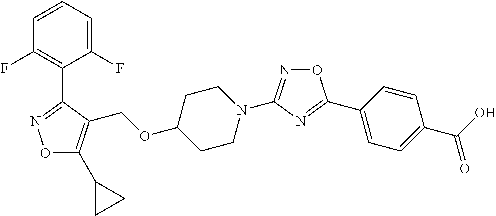 [SMILES: O=C(O)c1ccc(-c2nc(N3CCC(OCc4c(-c5c(F)cccc5F)noc4C4CC4)CC3)no2)cc1] | MS m/z 523.2 (M + 1); |
| 11 | 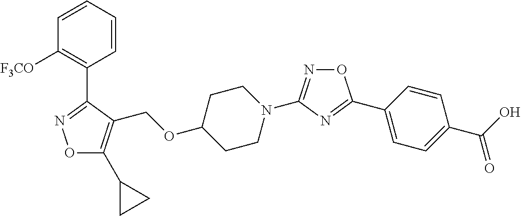 [SMILES: O=C(O)c1ccc(-c2nc(N3CCC(OCc4c(-c5ccccc5OC(F)(F)F)noc4C4CC4)CC3)no2)cc1] | MS m/z 555.2 (M + 1); |
| 12 | 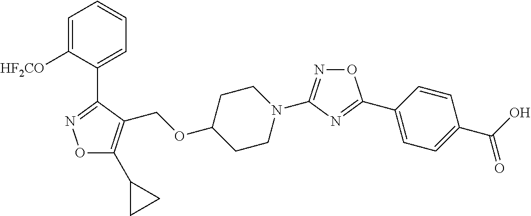 | MS (m/z) 553.1 (M + 1), 575 (M + 23). |

Assay Description

Human GST-FXR LBD Co-activator interaction Assay.

The FXR HTRF assay is a biochemical assay measuring the interaction between FXR and a coactivator protein (SRC1). The ligand-induced interaction with a coactivator protein is a critical step in transcriptional activation by FXR. Thus, this is an assay designed to measure FXR agonist activity of compounds.

Recombinant human Farnesoid X Receptor (FXR) ligand binding domain (amino acids 193-472) fused to glutathione S-transferase (GST) purified protein (GST-FXR LBD) was purchased (Invitrogen). The ligand-dependent interaction between GST-FXR LBD and a peptide derived from Steroid Receptor Coactivator-1 (SRC-1) was monitored by Fluorescence Resonance Energy Transfer (FRET). GST-FXR LBD was mixed with a biotin-labeled SRC-1 peptide (Sequence: Biotin-CPSSHSSLTERHKILHRLLQEG-SPS-CONH2, American Peptide) in assay buffer (50 mM Tris HCl, pH 7.4, 50 mM NaCl, 1 mM TCEP and 0.2% bovine serum albumen) and plated in 384 black Proxi plates (Greiner Bio-One). Test compounds (in DMSO solution) and detection reagents (anti-GST-Cryptate labeled antibody and Streptavidin-XL665 conjugate; CisBio) were added in assay buffer containing 50 mM KF. Plates are incubated at room temperature in the dark for 2.5 hours before reading on an Envision (PerkinElmer) at 665 nm and 590 nm. The HTRF assay results were calculated from the 665 nm/590 nm ratio (ratio=(A665 nm/A590 nm)×10⁴) and expressed in Delta F %=(Ratiosample−Rationegative)/Rationegative×100.

A negative control (without Streptavidin-XL665) was run with each assay and represented the background fluorescence. A reference FXR agonist, GW4064, was included in each experiment as positive control. The efficacy of each test compound was compared to that of GW4064. At each concentration, the relative activity of the test compound was expressed as Response %=(Rₛₐₘₚₗₑ−R_{DMSO})/(Rₚₒₛᵢₜᵢᵥₑ−R_{DMSO}), where Rₛₐₘₚₗₑ is the HTRF response, (expressed in Delta F %) for the test compound, Rₚₒₛᵢₜᵢᵥₑ is the maximal response for GW4064 at saturating concentrations, and R_{DMSO} is the response for DMSO control. The EC₅₀ values were calculated using GraphPad Prism (GraphPad Software) using non-linear regression curve fit (log(agonist) vs. response−variable slope (four parameters)).

Table 1 summarizes EC₅₀ values for the compounds of the invention in human GST-FXR LBD co-activator interaction assay.

TABLE 1

| | FXR Co-activator |
|---|---|
| | interaction assay |
| Example | (EC₅₀, uM) |
| 1A | 2.38 |
| 1B | 0.09 |
| 2A | 2.02 |
| 2B | 0.128 |
| 3A | 0.946 |
| 3B | 0.275 |
| 4A | 0.812 |
| 4B | 0.114 |
| 5B | 0.06 |
| 6A | 1.62 |
| 6B | 0.823 |
| 7A | 1.59 |
| 7B | 1.56 |
| 8A | 2.95 |
| 8B | 0.105 |
| 9A | 0.463 |
| 9B | 0.128 |
| 10 | 0.934 |
| 11 | 0.587 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1. A compound having Formula I: 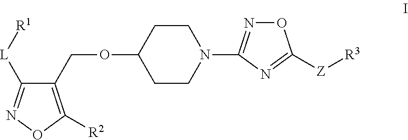
wherein Z is phenyl, C₅₋₇ cycloalkyl or 5-10 membered monocyclic or bicyclic heteroaryl containing 1-2 heteroatoms selected from N, O and S; each of which is optionally substituted with 1-2 R⁶ radicals selected from halogen, C₁₋₆ alkyl, haloC₁₋₆ alkyl, C₁₋₆ alkoxy, haloC₁₋₆ alkoxy, cyclopropyl or NR⁴R⁵;
L is a bond, C₁₋₄ alkylene or C₁₋₄alkylene-O—;
R¹ is phenyl substituted with 1-3 R^{1a}; or R¹ is C₃₋₈ cycloalkyl optionally substituted with 1-3 R^{1a} or phenyl;
R^{1a} is halogen, C₁₋₆ alkyl, haloC₁₋₆ alkyl, C₁₋₆ alkoxy or haloC₁₋₆alkoxy;
R² is C₁₋₃ alkyl, haloC₁₋₃ alkyl or cyclopropyl optionally substituted with C₁₋₃ alkyl or haloC₁₋₃ alkyl;
R³ is —X—CO₂R⁴, hydroxyC₁₋₆ alkyl, CONR⁴R⁵, CONR(CR₂)₁₋₄CO₂R⁴, CONR(CR₂)₁₋₄ SO₃R⁵, cyano, tetrazolyl or SO₂NR⁴R⁵; and
R⁴ and R⁵ are independently hydrogen or C₁₋₆ alkyl; and
X is a bond or C₁₋₄ alkylene; or
a stereoisomer, enantiomer, a pharmaceutically acceptable salt or an amino acid conjugate thereof.

2. The compound of claim 1, wherein L is a bond.

3. The compound of claim 1, wherein R² is cyclopropyl.

4. The compound of claim 1, wherein said compound is of Formula II: 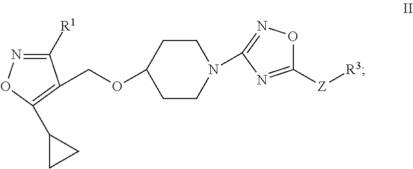
wherein Z is phenyl, thienyl, cyclopentyl, cyclohexyl or pyridyl, each of which is optionally substituted with 1-2 R⁶ radicals selected from halogen, C₁₋₆ alkyl, haloC₁₋₆ alkyl, C₁₋₆ alkoxy, haloC₁₋₆alkoxy, cyclopropyl or NR⁴R⁵;
R¹ is phenyl substituted with 1-2 R^{1a};
R^{1a} is halogen, C₁₋₆ alkyl, haloC₁₋₆ alkyl, C₁₋₆ alkoxy or haloC₁₋₆alkoxy;
R³ is —X—CO₂R⁴, hydroxyC₁₋₆ alkyl, CONR⁴R⁵, CONR(CR₂)₁₋₄CO₂R⁴, CONR(CR₂)₁₋₄ SO₃R⁵, cyano or tetrazolyl; and
R⁴ and R⁵ are independently hydrogen or C₁₋₆ alkyl; or
a stereoisomer, enantiomer, a pharmaceutically acceptable salt or an amino acid conjugate thereof.

5. The compound of claim 1, wherein said compound has a formula selected from the group consisting of: 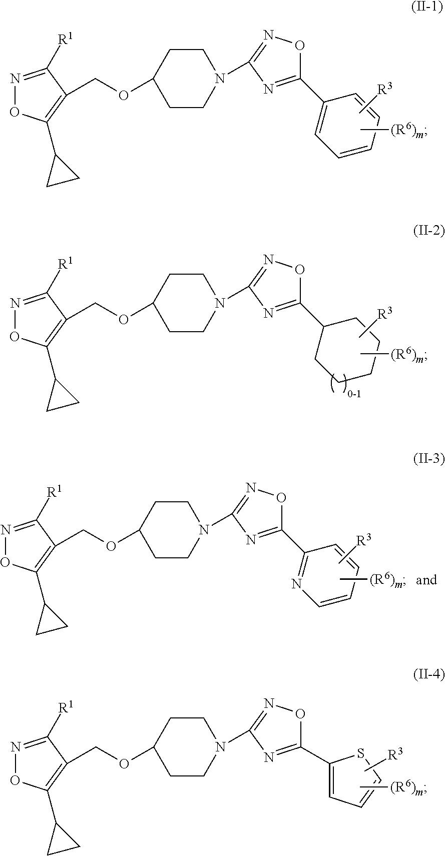
wherein R¹ is phenyl substituted with 1-2 R^{1a};
R^{1a} is halogen, C₁₋₆ alkyl, haloC₁₋₆ alkyl, C₁₋₆ alkoxy or haloC₁₋₆alkoxy;
R³ is —X—CO₂R⁴, hydroxyC₁₋₆ alkyl, CONR⁴R⁵, CONR(CR₂)₁₋₄CO₂R⁴, CONR(CR₂)₁₋₄ SO₃R⁵, cyano or tetrazolyl;
R⁴ and R⁵ are independently hydrogen or C₁₋₆ alkyl;
R⁶ is halogen, C₁₋₆ alkyl, haloC₁₋₆ alkyl, C₁₋₆ alkoxy or haloC₁₋₆alkoxy; and
m is 0-2; or
a stereoisomer, enantiomer, a pharmaceutically acceptable salt or an amino acid conjugate thereof.

6. The compound of claim 5, wherein m is 0-2; and R⁶ is selected from fluoro, chloro, bromo, methyl, methoxy, trifluoromethyl or trifluoromethoxy.

7. The compound of claim 1, wherein R^{1a} is selected from halo, methyl, trifluoromethyl, trifluoromethoxy, difluoromethoxy or methoxy.

8. The compound of claim 1, wherein R¹ is phenyl optionally substituted with 2,6-difluoro, 2-6-dichloro, trifluoromethyl, trifluoromethoxy or difluoromethoxy.

9. The compound of claim 1, R³ is X—CO₂R⁴; X is a bond and R⁴ is hydrogen or C₁₋₆ alkyl.

10. The compound of claim 1, wherein said compound is selected from
methyl 4-{3-[4-({5-cyclopropyl-3-[2-(trifluoromethoxy)phenyl]-1,2-oxazol-4-yl}methoxy)piperidin-1-yl]-1,2,4-oxadiazol-5-yl}benzoate;
4-{3-[4-({5-cyclopropyl-3-[2-(trifluoromethoxy)phenyl]-1,2-oxazol-4-yl}methoxy)piperidin-1-yl]-1,2,4-oxadiazol-5-yl}benzoic acid;
methyl 3-{3-[4-({5-cyclopropyl-3-[2-(trifluoromethoxy)phenyl]-1,2-oxazol-4-yl}methoxy)piperidin-1-yl]-1,2,4-oxadiazol-5-yl}benzoate;
3-{3-[4-({5-cyclopropyl-3-[2-(trifluoromethoxy)phenyl]-1,2-oxazol-4-yl}methoxy)piperidin-1-yl]-1,2,4-oxadiazol-5-yl}benzoic acid;
methyl 5-{3-[4-({5-cyclopropyl-3-[2-(trifluoromethoxy)phenyl]-1,2-oxazol-4-yl}methoxy)piperidin-1-yl]-1,2,4-oxadiazol-5-yl}thiophene-2-carboxylate;
5-{3-[4-({5-cyclopropyl-3-[2-(trifluoromethoxy)phenyl]-1,2-oxazol-4-yl}methoxy)piperidin-1-yl]-1,2,4-oxadiazol-5-yl}thiophene-2-carboxylic acid;
methyl 4-[3-(4-{[5-cyclopropyl-3-(2,6-dichlorophenyl)-1,2-oxazol-4-yl]methoxy}piperidin-1-yl)-1,2,4-oxadiazol-5-yl]benzoate;
4-[3-(4-{[5-cyclopropyl-3-(2,6-dichlorophenyl)-1,2-oxazol-4-yl]methoxy}piperidin-1-yl)-1,2,4-oxadiazol-5-yl]benzoic acid;
methyl 3-[3-(4-{[5-cyclopropyl-3-(2,6-dichlorophenyl)-1,2-oxazol-4-yl]methoxy}piperidin-1-yl)-1,2,4-oxadiazol-5-yl]benzoate;
3-[3-(4-{[5-cyclopropyl-3-(2,6-dichlorophenyl)-1,2-oxazol-4-yl]methoxy}piperidin-1-yl)-1,2,4-oxadiazol-5-yl]benzoic acid;
methyl 4-[3-(4-{[5-cyclopropyl-3-(2,6-dichlorophenyl)-1,2-oxazol-4-yl]methoxy}piperidin-1-yl)-1,2,4-oxadiazol-5-yl]cyclohexane-1-carboxylate;
4-[3-(4-{[5-cyclopropyl-3-(2,6-dichlorophenyl)-1,2-oxazol-4-yl]methoxy}piperidin-1-yl)-1,2,4-oxadiazol-5-yl]cyclohexane-1-carboxylic acid;
methyl-3-[3-(4-{[5-cyclopropyl-3-(2,6-dichlorophenyl)-1,2-oxazol-4-yl]methoxy}piperidin-1-yl)-1,2,4-oxadiazol-5-yl]cyclopentane-1-carboxylate;
methyl (1R,3S)-3-[3-(4-{[5-cyclopropyl-3-(2,6-dichlorophenyl)-1,2-oxazol-4-yl]methoxy}piperidin-1-yl)-1,2,4-oxadiazol-5-yl]cyclopentane-1-carboxylate;
methyl (1R,3R)-3-[3-(4-{[5-cyclopropyl-3-(2,6-dichlorophenyl)-1,2-oxazol-4-yl]methoxy}piperidin-1-yl)-1,2,4-oxadiazol-5-yl]cyclopentane-1-carboxylate;
methyl (1S,3S)-3-[3-(4-{[5-cyclopropyl-3-(2,6-dichlorophenyl)-1,2-oxazol-4-yl]methoxy}piperidin-1-yl)-1,2,4-oxadiazol-5-yl]cyclopentane-1-carboxylate;
methyl (1S,3R)-3-[3-(4-{[5-cyclopropyl-3-(2,6-dichlorophenyl)-1,2-oxazol-4-yl]methoxy}piperidin-1-yl)-1,2,4-oxadiazol-5-yl]cyclopentane-1-carboxylate;
3-[3-(4-{[5-cyclopropyl-3-(2,6-dichlorophenyl)-1,2-oxazol-4-yl]methoxy}piperidin-1-yl)-1,2,4-oxadiazol-5-yl]cyclopentane-1-carboxylic acid;
3S)-3-[3-(4-{[5-cyclopropyl-3-(2,6-dichlorophenyl)-1,2-oxazol-4-yl]methoxy}piperidin-1-yl)-1,2,4-oxadiazol-5-yl]cyclopentane-1-carboxylic acid;
3R)-3-[3-(4-{[5-cyclopropyl-3-(2,6-dichlorophenyl)-1,2-oxazol-4-yl]methoxy}piperidin-1-yl)-1,2,4-oxadiazol-5-yl]cyclopentane-1-carboxylic acid;
3S)-3-[3-(4-{[5-cyclopropyl-3-(2,6-dichlorophenyl)-1,2-oxazol-4-yl]methoxy}piperidin-1-yl)-1,2,4-oxadiazol-5-yl]cyclopentane-1-carboxylic acid;
3R)-3-[3-(4-{[5-cyclopropyl-3-(2,6-dichlorophenyl)-1,2-oxazol-4-yl]methoxy}piperidin-1-yl)-1,2,4-oxadiazol-5-yl]cyclopentane-1-carboxylic acid;
methyl 5-[3-(4-{[5-cyclopropyl-3-(2,6-dichlorophenyl)-1,2-oxazol-4-yl]methoxy}piperidin-1-yl)-1,2,4-oxadiazol-5-yl]thiophene-2-carboxylate;
5-[3-(4-{[5-cyclopropyl-3-(2,6-dichlorophenyl)-1,2-oxazol-4-yl]methoxy}piperidin-1-yl)-1,2,4-oxadiazol-5-yl]thiophene-2-carboxylic acid;
methyl 6-[3-(4-{[5-cyclopropyl-3-(2,6-dichlorophenyl)-1,2-oxazol-4-yl]methoxy}piperidin-1-yl)-1,2,4-oxadiazol-5-yl]pyridine-2-carboxylate;
6-[3-(4-{[5-cyclopropyl-3-(2,6-dichlorophenyl)-1,2-oxazol-4-yl]methoxy}piperidin-1-yl)-1,2,4-oxadiazol-5-yl]pyridine-2-carboxylic acid;
4-[3-(4-{[5-cyclopropyl-3-(2,6-difluorophenyl)-1,2-oxazol-4-yl]methoxy}piperidin-1-yl)-1,2,4-oxadiazol-5-yl]benzoic acid;
4-{3-[4-({5-cyclopropyl-3-[2-(trifluoromethyl)phenyl]-1,2-oxazol-4-yl}methoxy)piperidin-1-yl]-1,2,4-oxadiazol-5-yl}benzoic acid; and
4-{3-[4-({5-cyclopropyl-3-[2-(difluoromethoxy)phenyl]-1,2-oxazol-4-yl}methoxy)piperidin-1-yl]-1,2,4-oxadiazol-5-yl}benzoic acid; or
a stereoisomer, enantiomer, a pharmaceutically acceptable salt or an amino acid conjugate thereof.

11. A pharmaceutical composition comprising a therapeutically effective amount of a compound of claim 1 and a pharmaceutically acceptable carrier.

12. A combination comprising a therapeutically effective amount of a compound according to claim 1, and a second therapeutic agent being useful in the treatment of cholestasis, intrahepatic cholestatis, estrogen-induced cholestasis, drug-induced cholestasis, cholestasis of pregnancy, parenteral nutrition-associated cholestasis, primary biliary cirrhosis (PBC), primary sclerosing cholangistis (PSC), progressive familiar cholestatis (PFIC), non-alcoholic fatty liver disease (NAFLD), non-alcoholic steatohepatitis (NASH), drug-induced bile duct injury, gallstones, liver cirrhosis, alcohol-induced cirrhosis, cystic fibrosis, bile duct obstruction, cholelithiasis, liver fibrosis, dyslipidemia, atherosclerosis, diabetes, diabetic nephropathy, colitis, newborn jaundice, prevention of kernicterus, venocclusive disease, portal hypertension, metabolic syndrome, hypercholesterolemia, intestinal bacterial overgrowth, or erectile dysfunction.

13. A method for treating a condition mediated by FXR in a subject suffering therefrom, comprising administering to the subject a therapeutically effective amount of a compound of claim 1, or a pharmaceutical composition thereof, and optionally in combination with a second therapeutic agent.

14-16. (canceled)

17. A compound having Formula III 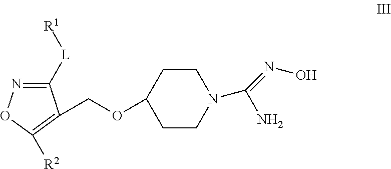
wherein R¹, R² and L are as defined in claim 1; or pharmaceutically acceptable salts thereof.

18. The compound of claim 17, wherein R² is or cyclopropyl.

19. A process for preparing a compound of Formula I according to claim 1, comprising reacting a compound of Formula III: 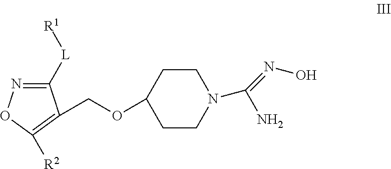
with a compound of Formula R³—Z—C(O)OH to form a compound of Formula IV: 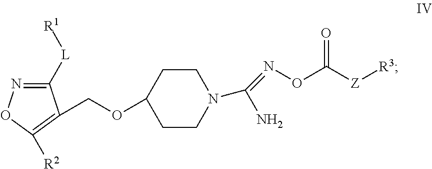
and
converting the compound of Formula IV to a compound of Formula I;
wherein R¹, R², L and Z are as defined in claim 1;
R³ is —X—CO₂R⁴ wherein X is a bond or methylene;
R⁴ is C₁₋₆ alkyl;
and optionally, converting a compound of Formula I, wherein the substituents have the meaning as defined in claim 1, into another compound of Formula I as defined in claim 1; and
recovering the resulting compound of Formula I in free form or as a salt; and optionally converting the compound of Formula I obtained in free form into a desired salt, or an obtained salt into the free form.

20. The method of claim 13, wherein said condition is cholestasis, intrahepatic cholestatis, estrogen-induced cholestasis, drug-induced cholestasis, cholestasis of pregnancy, parenteral nutrition-associated cholestasis, primary biliary cirrhosis (PBC), primary sclerosing cholangistis (PSC), progressive familiar cholestatis (PFIC), non-alcoholic fatty liver disease (NAFLD), non-alcoholic steatohepatitis (NASH), drug-induced bile duct injury, gallstones, liver cirrhosis, alcohol-induced cirrhosis, cystic fibrosis, bile duct obstruction, cholelithiasis, liver fibrosis, dyslipidemia, atherosclerosis, diabetes, diabetic nephropathy, colitis, newborn jaundice, prevention of kernicterus, venocclusive disease, portal hypertension, metabolic syndrome, hypercholesterolemia, intestinal bacterial overgrowth, or erectile dysfunction.
